(19) United States
(12) Patent Application Publication
(10) Pub. No.: US 2020/0385754 A1
(43) Pub. Date: Dec. 10, 2020

(54) ENGINEERED UBIQUITOUS CHROMATIN OPENING ELEMENTS AND USES THEREOF

(71) Applicant: Integrity BioSolutions, LLC, San Diego, CA (US)

(72) Inventor: Jason KAHANA, San Diego, CA (US)

(21) Appl. No.: 16/769,522

(22) PCT Filed: Dec. 6, 2018
(86) PCT No.: PCT/US2018/064339

§ 371 Date: Jun. 3, 2020

Related U.S. Application Data
(60) Provisional application No. 62/595,811, filed on Dec. 7, 2017.

(51) Int. Cl. C12N 15/85

(52) U.S. Cl. CPC C12N 15/85; C12N 2830/002; C12N 2830/60; C12N 2830/50

(57) ABSTRACT

Disclosed herein are polynucleic acid molecules, plasmids, vectors, compositions, methods, and kits for expressing a target protein. In some instances, also described herein are polynucleic acid molecules, plasmids, vectors, compositions, methods, and kits for enhancing the expression of a target protein.

### CROSS-REFERENCE

This application claims the benefit of U.S. Provisional Application No. 62/595,811, filed Dec. 7, 2017, which is incorporated herein by reference in its entirety.

### SEQUENCE LISTING

The instant application contains a Sequence Listing which has been submitted electronically in ASCII format and is hereby incorporated by reference in its entirety. Said ASCII copy, created on Dec. 6, 2018, is named 53513-701_601_SL.txt and is 39,529 bytes in size.

### SUMMARY OF THE DISCLOSURE

Disclosed herein, in certain embodiments, is a ubiquitous chromatin opening element (UCOE) for expressing and/or enhancing expression of a target protein of interest. Also disclosed herein include plasmids, vectors, compositions, methods, and kits for enhancing the expression of a target protein.

Disclosed herein, in certain embodiments, is an isolated polynucleotide comprising: an extended methylation-free CpG island encompassing dual divergently transcribed promoters; a target gene of interest adjacent to the extended methylation-free CpG island; a polyadenylation signal located at the 3′ terminus of the target gene of interest; and optionally one or more selectable markers; wherein the GC content of the extended methylation-free CpG island over a 200 bp range is from about 62% to about 88%. In some embodiments, the GC content of the extended methylation-free CpG island over a 200 bp range is from about 62.5% to about 87.5%, about 63% to about 87%, about 65% to about 85%, about 70% to about 80%, or about 75% to about 80%. In some embodiments, at least one of the dual divergently transcribed promoters comprises a constitutive promoter. In some embodiments, at least one of the dual divergently transcribed promoters comprises an inducible promoter. In some embodiments, at least one of the dual divergently transcribed promoters comprises a eukaryotic promoter. In some embodiments, at least one of the dual divergently transcribed promoters comprises a prokaryotic promoter. In some embodiments, at least one of the dual divergently transcribed promoters comprises a viral promoter. In some embodiments, at least one of the dual divergently transcribed promoters comprises a CMV promoter. In some embodiments, at least one of the dual divergently transcribed promoters comprises a HSV TK promoter. In some embodiments, the extended methylation-free CpG island comprises at least 80%, 85%, 90%, 95%, 96%, 97%, 98%, or 99% sequence identity to SEQ ID NO: 1. In some embodiments, the extended methylation-free CpG island comprises or consists of SEQ ID NO: 1. In some embodiments, if present, at least one of the one or more selectable markers is located adjacent to the extended methylation-free CpG island but at the opposing terminus from the target gene of interest. In some embodiments, if present, at least one of the one or more selectable markers is located between the extended methylation-free CpG island and the target gene of interest. In some embodiments, if present, at least one of the one or more selectable markers is located proximal to the polyadenylation signal. In some embodiments, the selectable marker is more than 2000 bp from the proximal end of the polyadenylation signal. In some embodiments, at least one of the one or more selectable markers is in a separate vector. In some embodiments, at least one of the one or more selectable markers is an antibiotic resistant gene. In some embodiments, at least one of the one or more selectable markers is a selectable marker for a mammalian vector. In some embodiments, the selectable marker for a mammalian vector comprises ada, BSD, Ble, Pac, neo, hisD, GS, dhfr, codA, or Hph. In some embodiments, the polynucleotide further comprises a promoter. In some embodiments, the promoter is an exogenous promoter. In some embodiments, the promoter is located adjacent to the 5′ terminus of the target gene of interest. In some embodiments, the promoter is SP6 promoter.

Disclosed herein, in certain embodiments, is an isolated polynucleotide comprising: an extended methylation-free CpG island encompassing dual divergently transcribed promoters; a target gene of interest adjacent to the extended methylation-free CpG island; a polyadenylation signal located at the 3′ terminus of the target gene of interest; and optionally one or more selectable markers; wherein the GC content of the extended methylation-free CpG island is higher than 62%. In some embodiments, the GC content of the extended methylation-free CpG island is higher than 63%, 64%, 65%, 70%, 75%, 80%, or 85%. In some embodiments, the GC content of the extended methylation-free CpG island is over a 200 bp range. In some embodiments, at least one of the dual divergently transcribed promoters comprises a constitutive promoter. In some embodiments, at least one of the dual divergently transcribed promoters comprises an inducible promoter. In some embodiments, at least one of the dual divergently transcribed promoters comprises a eukaryotic promoter. In some embodiments, at least one of the dual divergently transcribed promoters comprises a prokaryotic promoter. In some embodiments, at least one of the dual divergently transcribed promoters comprises a viral promoter. In some embodiments, at least one of the dual divergently transcribed promoters comprises a CMV promoter. In some embodiments, at least one of the dual divergently transcribed promoters comprises a HSV TK promoter. In some embodiments, the extended methylation-free CpG island comprises at least 80%, 85%, 90%, 95%, 96%, 97%, 98%, or 99% sequence identity to SEQ ID NO: 1. In some embodiments, the extended methylation-free CpG island comprises or consists of SEQ ID NO: 1. In some embodiments, if present, at least one of the one or more selectable markers is located adjacent to the extended methylation-free CpG island but at the opposing terminus from the target gene of interest. In some embodiments, if present, at least one of the one or more selectable markers is located between the extended methylation-free CpG island and the target gene of interest. In some embodiments, if present, at least one of the one or more selectable markers is located proximal to the polyadenylation signal. In some embodiments, the selectable marker is more than 2000 bp from the proximal end of the polyadenylation signal. In some embodiments, at least one of the one or more selectable markers is in a separate vector. In some embodiments, at least one of the one or more selectable markers is an antibiotic resistant gene. In some embodiments, at least one of the one or more selectable markers is a selectable marker for a mammalian vector. In some embodiments, the selectable marker for a mammalian vector comprises ada, BSD, Ble, Pac, neo, hisD, GS, dhfr, codA, or Hph. In some embodiments, the polynucleotide further comprises a promoter. In some embodiments, the promoter is an exogenous promoter. In some embodiments, the promoter is located adjacent to the 5′ terminus of the target gene of interest. In some embodiments, the promoter is SP6 promoter.

Disclosed herein, in certain embodiments, is an isolated vector comprising a polynucleotide described above. In some embodiments, the isolated vector comprises two or more selectable markers. In some embodiments, the isolated vector comprises at least 50%, 60%, 70%, 80%, 90%, 95%, or 99% sequence identity to a sequence selected from SEQ ID NOs: 2, 3, or 5. In some embodiments, the isolated vector comprises or consists of SEQ ID NO: 2, 3, or 5.

Disclosed herein, in certain embodiments, is a recombinant engineered host cell comprising an isolated polynucleotide described above or an isolated vector described above.

Disclosed herein, in certain embodiments, is an isolated vector comprising a polynucleotide comprising at least 80%, 85%, 90%, 95%, 96%, 97%, 98%, or 99% sequence identity to SEQ ID NO: 1.

Disclosed herein, in certain embodiments, is an isolated vector comprising at least 50%, 60%, 70%, 80%, 90%, 95%, or 99% sequence identity to a sequence selected from SEQ ID NOs: 2, 3, or 5.

Disclosed herein, in certain embodiments, is a method of enhancing the expression of a target protein, comprising: (a) contacting a host cell with an isolated polynucleotide described above or an isolated vector described above, wherein the target gene of interest encodes the target protein; and (b) culturing the host cell at a sufficient condition wherein the host cell expresses the target protein, thereby inducing an enhanced expression of the target protein. In some embodiments, the host cell is a eukaryotic host cell. In some embodiments, the host cell is from CHO DG44 cell line, CHO-S cell line, CHO-K1 cell line, Flp-In-CHO cell line, FreeStyle CHO-S cell line, GS-CHO cell line, 293T cell line, 293A cell line, 293FT cell line, 293F cell line, 293 H cell line, A549 cell line, MDCK cell line, HepaRG cell line, T-REx Jurkat cell line, Per.C6 cell line, T-REx-293 cell line, T-REx-CHO cell line, or T-REx-HeLa cell line. In some embodiments, the sufficient condition is a serum-free condition.

In certain embodiments, described herein is a kit comprising an isolated polynucleotide described above or an isolated vector described above.

### BRIEF DESCRIPTION OF THE DRAWINGS

Various aspects of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings of which:

FIG. 1A-FIG. 1D illustrate vector maps of exemplary isolated vector sequences described herein. FIG. 1A shows the vector map of SEQ ID NO: 2. FIG. 1B shows the vector map of SEQ ID NO: 3. FIG. 1C shows the vector map of SEQ ID NO: 5. FIG. 1D shows the vector map of SEQ ID NO: 4.

FIG. 2A shows an illustrative phase contrast image of cells transfected with Plasmid 1.

FIG. 2B shows an illustrative GFP fluorescence of cells transfected with Plasmid 1.

FIG. 3A shows an illustrative phase contrast image of cells transfected with Plasmid 2.

FIG. 3B shows an illustrative GFP fluorescence of cells transfected with Plasmid 2.

FIG. 4 shows illustrative GFP fluorescence of HEK293 cells transfected with Plasmid 3.

FIG. 5 shows illustrative GFP expression taken on Day 107 from date of transfection.

### DETAILED DESCRIPTION OF THE DISCLOSURE

Recombinant protein expression systems are based on the introduction of a foreign gene in an expression vector into prokaryotic or eukaryotic cells, as an additional episome or integrated part of the host cell genome. The production of foreign proteins is then achieved by efficient transcription and translation by host cell machineries. Commonly used hosts are bacterial, yeast, insect and mammalian cells. Of these, mammalian expression systems enable the production of recombinant proteins that possess relevant post-translational modifications and exhibit high enzymatic activity.

For efficient expression of a target protein in mammalian cells, factors that modulate efficiency include, e.g., expression vectors, appropriate host cells, and gene transfer reagents. In the last two decades, a variety of expression vectors have been developed for propagating and expressing covalently linked genes in different types of host cells. Although many cultured mammalian cells of different origins are used for this purpose, few host cells are suitable for large-scale production. Gene transfer reagents further modulate expression efficiency.

In some embodiments, described herein is a ubiquitous chromatin opening element (UCOE), also known as an extended methylation-free CpG island encompassing dual divergently transcribed promoters, for expressing and/or enhancing expression of a target protein of interest. In some cases, also described herein are isolated polynucleotides, vectors, and host cells that comprise an extended methylation-free CpG island encompassing dual divergently transcribed promoters. In additional cases, described herein are methods of using the extended methylation-free CpG island encompassing dual divergently transcribed promoters for expressing and/or enhancing expression of a target protein of interest.

### Ubiquitous Chromatin Opening Elements

Ubiquitous chromatin opening element (UCOE), also known as an extended methylation-free CpG island encompassing dual divergently transcribed promoters, are elements that open chromatin or maintain chromatin in an open state and facilitates reproducible expression of an operably-linked gene in cells. In some cases, an “extended” methylation-free CpG island encompasses a methylation-free CpG island that extends across a region encompassing more than one transcriptional start site and/or extends for more than 300 bp, more than 500 bp, more than 1000bp, more than 1500 bp, more than 2000 bp, more than 2500 bp, or more than 3000 bp.

In some embodiments, the GC content of an extended methylation-free CpG island described herein over a 200 bp range is from about 62% to about 88%. In some instances, the GC content of an extended methylation-free CpG island described herein over a 200 bp range is from about 62.5% to about 87.5%, about 63% to about 87%, about 63% to about 85%, about 63% to about 80%, about 63% to about 75%, about 63% to about 70%, about 65% to about 85%, about 65% to about 80%, about 65% to about 75%, about 65% to about 70%, about 70% to about 85%, about 70% to about 80%, or about 75% to about 80%. In some instances, the GC content of an extended methylation-free CpG island described herein over a 200 bp range is from about 62.5% to about 87.5%, about 63% to about 87%, about 65% to about 85%, about 70% to about 80%, or about 75% to about 80%.

In some embodiments, the GC content of an extended methylation-free CpG island described herein is higher than 63%, 64%, 65%, 70%, 75%, 80%, or 85%. In some instances, the GC content of an extended methylation-free CpG island described herein is higher than 63%. In some instances, the GC content of an extended methylation-free CpG island described herein is higher than 64%. In some instances, the GC content of an extended methylation-free CpG island described herein is higher than 65%. In some instances, the GC content of an extended methylation-free CpG island described herein is higher than 66%. In some instances, the GC content of an extended methylation-free CpG island described herein is higher than 67%. In some instances, the GC content of an extended methylation-free CpG island described herein is higher than 68%. In some instances, the GC content of an extended methylation-free CpG island described herein is higher than 69%. In some instances, the GC content of an extended methylation-free CpG island described herein is higher than 70%. In some instances, the GC content of an extended methylation-free CpG island described herein is higher than 71%. In some instances, the GC content of an extended methylation-free CpG island described herein is higher than 72%. In some instances, the GC content of an extended methylation-free CpG island described herein is higher than 73%. In some instances, the GC content of an extended methylation-free CpG island described herein is higher than 74%. In some instances, the GC content of an extended methylation-free CpG island described herein is higher than 75%. In some instances, the GC content of an extended methylation-free CpG island described herein is higher than 76%. In some instances, the GC content of an extended methylation-free CpG island described herein is higher than 77%. In some instances, the GC content of an extended methylation-free CpG island described herein is higher than 78%. In some instances, the GC content of an extended methylation-free CpG island described herein is higher than 79%. In some instances, the GC content of an extended methylation-free CpG island described herein is higher than 80%. In some instances, the GC content of an extended methylation-free CpG island described herein is higher than 81%. In some instances, the GC content of an extended methylation-free CpG island described herein is higher than 82%. In some instances, the GC content of an extended methylation-free CpG island described herein is higher than 83%. In some instances, the GC content of an extended methylation-free CpG island described herein is higher than 84%. In some instances, the GC content of an extended methylation-free CpG island described herein is higher than 85%. In some cases, the GC content of the methylation-free CpG island is over a 200 bp range. In other cases, the GC content of the methylation-free CpG island is over a 500 bp range. In additional cases, the GC content of the methylation-free CpG island is over a 1000 bp range, a 1500 bp range, a 2000 bp range, or higher.

In some embodiments, an extended methylation-free CpG island described herein comprises at least 80%, 85%, 90%, 95%, 96%, 97%, 98%, or 99% sequence identity to SEQ ID NO: 1. In some instances, the extended methylation-free CpG island described herein comprises at least 80% sequence identity to SEQ ID NO: 1. In some instances, the extended methylation-free CpG island described herein comprises at least 85% sequence identity to SEQ ID NO: 1. In some instances, the extended methylation-free CpG island described herein comprises at least 90% sequence identity to SEQ ID NO: 1. In some instances, the extended methylation-free CpG island described herein comprises at least 91% sequence identity to SEQ ID NO: 1. In some instances, the extended methylation-free CpG island described herein comprises at least 92% sequence identity to SEQ ID NO: 1. In some instances, the extended methylation-free CpG island described herein comprises at least 93% sequence identity to SEQ ID NO: 1. In some instances, the extended methylation-free CpG island described herein comprises at least 94% sequence identity to SEQ ID NO: 1. In some instances, the extended methylation-free CpG island described herein comprises at least 95% sequence identity to SEQ ID NO: 1. In some instances, the extended methylation-free CpG island described herein comprises at least 96% sequence identity to SEQ ID NO: 1. In some instances, the extended methylation-free CpG island described herein comprises at least 97% sequence identity to SEQ ID NO: 1. In some instances, the extended methylation-free CpG island described herein comprises at least 98% sequence identity to SEQ ID NO: 1. In some instances, the extended methylation-free CpG island described herein comprises at least 99% sequence identity to SEQ ID NO: 1. In some instances, the extended methylation-free CpG island described herein comprises 100% sequence identity to SEQ ID NO: 1. In some instances, the extended methylation-free CpG island described herein consists of SEQ ID NO: 1.

In some embodiments, an extended methylation-free CpG island described herein comprises at least 80%, 85%, 90%, 95%, 96%, 97%, 98%, or 99% sequence identity to at least 200 contiguous bases of SEQ ID NO: 1. In some cases, the extended methylation-free CpG island described herein comprises at least 80%, 85%, 90%, 95%, 96%, 97%, 98%, or 99% sequence identity to at least 300 contiguous bases of SEQ ID NO: 1. In some cases, the extended methylation-free CpG island described herein comprises at least 80%, 85%, 90%, 95%, 96%, 97%, 98%, or 99% sequence identity to at least 500 contiguous bases of SEQ ID NO: 1. In some cases, the extended methylation-free CpG island described herein comprises at least 80%, 85%, 90%, 95%, 96%, 97%, 98%, or 99% sequence identity to at least 1000 contiguous bases of SEQ ID NO: 1. In some cases, the extended methylation-free CpG island described herein comprises at least 80%, 85%, 90%, 95%, 96%, 97%, 98%, or 99% sequence identity to at least 1250 contiguous bases of SEQ ID NO: 1. In some cases, the extended methylation-free CpG island described herein comprises at least 80%, 85%, 90%, 95%, 96%, 97%, 98%, or 99% sequence identity to at least 1500 contiguous bases of SEQ ID NO: 1. In some cases, the extended methylation-free CpG island described herein comprises at least 80%, 85%, 90%, 95%, 96%, 97%, 98%, or 99% sequence identity to at least 1750 contiguous bases of SEQ ID NO: 1. In some cases, the extended methylation-free CpG island described herein comprises at least 80%, 85%, 90%, 95%, 96%, 97%, 98%, or 99% sequence identity to at least 2000 contiguous bases of SEQ ID NO: 1. In some cases, the extended methylation-free CpG island described herein comprises at least 80%, 85%, 90%, 95%, 96%, 97%, 98%, or 99% sequence identity to at least 2250 contiguous bases of SEQ ID NO: 1. In some cases, the extended methylation-free CpG island described herein comprises at least 80%, 85%, 90%, 95%, 96%, 97%, 98%, or 99% sequence identity to at least 2500 contiguous bases of SEQ ID NO: 1.

In some embodiments, a polynucleotide described herein comprises an extended methylation-free CpG island comprising at least 80%, 85%, 90%, 95%, 96%, 97%, 98%, or 99% sequence identity to SEQ ID NO: 1.

### Target Genes of Interest

In certain embodiments, a target gene of interest described herein includes a sequence encoding a receptor, an enzyme, a ligand, a regulatory factor, a hormone, an antibody or antibody fragment, or a structural protein. In some instances, exemplary target genes of interest include sequences encoding nuclear proteins, cytoplasmic proteins, mitochondrial proteins, secreted proteins, membrane-associated proteins, serum proteins, viral antigens, bacterial antigens, protozoal antigens and parasitic antigens. In some cases, the target genes of interest include sequences encoding peptides, lipoproteins, glycoproteins, phosphoproteins, and nucleic acid (e.g., RNAs or antisense nucleic acids). Exemplary class of protein or polypeptide which can be encoded by the target gene sequence include, but are not limited to, hormones, growth factors, enzymes, clotting factors, apolipoproteins, receptors, erythropoietin, therapeutic antibodies or fragments thereof, drugs, oncogenes, tumor antigens, tumor suppressors, viral antigens, parasitic antigens, and bacterial antigens. In some cases, the target genes of interest include sequences encoding proinsulin, growth hormone, androgen receptors, insulin-like growth factor I, insulin-like growth factor II, insulin-like growth factor binding proteins, epidermal growth factor, transforming growth factor-a transforming growth factor-β, plate let-derived growth factor, angiogenesis factors (acidic fibroblast growth factor, basic fibroblast growth factor, vascular endothelial growth factor and angiogenin), matrix proteins (Type IV collagen, Type VII collagen, laminin), phenylalanine hydroxylase, tyrosine hydroxylase, oncoproteins (for example, those encoded by ras, fos, myc, erb, src, neu, sis, jun), HPV E6 or E7 oncoproteins, p53 protein, Rb protein, cytokine receptors, IL-1, IL-6, IL-8, and proteins from viral, bacterial and parasitic organisms which can be used to induce an immunological response, and other proteins of useful significance in the body. The choice of gene, to be incorporated, is only limited by the availability of the nucleic acid sequence encoding it. One skilled in the art will readily recognize that as more proteins and polypeptides become identified they can be integrated into the polynucleotide of the present disclosure and expressed.

### Selectable Markers

In certain embodiments, an isolated polynucleotide described herein optionally comprises one or more selectable markers. In some cases, at least one of the one or more selectable markers is located adjacent to the extended methylation-free CpG island but at the opposing terminus from the target gene of interest. In other cases, at least one of the one or more selectable markers is located between the extended methylation-free CpG island and the target gene of interest. In additional cases, at least one of the one or more selectable markers is located proximal to the polyadenylation signal.

In some embodiments, an isolated polynucleotide described herein comprises two or more selectable markers. In some instances, the first selectable marker is located adjacent to the extended methylation-free CpG island but at the opposing terminus from the target gene of interest, and the second selectable marker is located proximal to the polyadenylation signal, between the extended methylation-free CpG island and the target gene of interest, proximal to the extended methylation-free CpG island, or adjacent to the first selectable marker. In some cases, the first selectable marker is located adjacent to the extended methylation-free CpG island but at the opposing terminus from the target gene of interest and the second selectable marker is located proximal to the target gene of interest but at the opposing terminus from the first selectable marker with respect to the extended methylation-free CpG island.

In some embodiments, the selectable marker is an antibiotic resistant gene. Exemplary antibiotic resistant genes include, but are not limited to, ampicillin, chloramphenicol, kanamycin, tetracycline, polymyxin B, erythromycin, carbenicillin, streptomycin, spectinomycin, blasticidin S deaminases (Bsr, BSD), bleomycin-binding protein (Ble), Neomycin phosphotransferase (neo), puromycin N-acetyltransferase (Pac), zeocin (Sh bla), and hygromycin B phosphotransferase (Hph). In some cases, the selectable marker is a eukaryotic antibiotic resistant gene. In some cases, the selectable marker is blasticidin S deaminases (Bsr, BSD), bleomycin-binding protein (Ble), Neomycin phosphotransferase (neo), puromycin N-acetyltransferase (Pac), zeocin (Sh bla), and hygromycin B phosphotransferase (Hph).

In some instances, at least one of the one or more selectable markers is an antibiotic resistant gene. In some cases, at least one of the one or more selectable markers is a eukaryotic antibiotic resistant gene. In some cases, at least one of the one or more selectable markers is blasticidin S deaminases (Bsr, BSD), bleomycin-binding protein (Ble), Neomycin phosphotransferase (neo), puromycin N-acetyltransferase (Pac), zeocin (Sh bla), and hygromycin B phosphotransferase (Hph).

In some embodiments, the isolated polynucleotide optionally comprises two or more selectable markers. In some cases, the first selectable marker is located adjacent to the extended methylation-free CpG island but at the opposing terminus from the target gene of interest, between the extended methylation-free CpG island and the target gene of interest, or proximal to the polyadenylation signal.

In some instances, the first selectable marker is an antibiotic resistant gene. In some cases, the first selectable marker is a eukaryotic antibiotic resistant gene. In some cases, the first selectable marker is blasticidin S deaminases (Bsr, BSD), bleomycin-binding protein (Ble), Neomycin phosphotransferase (neo), puromycin N-acetyltransferase (Pac), zeocin (Sh bla), and hygromycin B phosphotransferase (Hph).

In some cases, the second selectable marker is located proximal to the polyadenylation signal, between the extended methylation-free CpG island and the target gene of interest, proximal to the extended methylation-free CpG island, or adjacent to the first selectable marker.

In some cases, the second selectable marker is an antibiotic resistant gene. In some cases, the second selectable marker is a prokaryotic selectable marker. In some cases, the second selectable marker is ampicillin, chloramphenicol, kanamycin, tetracycline, polymyxin B, erythromycin, bleomycin, carbenicillin, streptomycin, or spectinomycin.

In some embodiments, the selectable marker is a selectable marker for mammalian expression vectors. Exemplary selectable markers include, but are not limited to, adenine deaminase (ada), blasticidin S deaminases (Bsr, BSD), bleomycin-binding protein (Ble), Neomycin phosphotransferase (neo), histidinol dehydrogenase (hisD), glutamine synthetase (GS) (also known as glutamine ammonia ligase or GLUL), dihydrofolate reductase (dhfr), cytosine deaminase (codA), puromycin N-acetyltransferase (Pac), and hygromycin B phosphotransferase (Hph). In some instances, the selectable marker is adenine deaminase (ada), blasticidin S deaminases (Bsr, BSD), bleomycin-binding protein (Ble), Neomycin phosphotransferase (neo), histidinol dehydrogenase (hisD), glutamine synthetase (GS), dihydrofolate reductase (dhfr), cytosine deaminase (codA), puromycin N-acetyltransferase (Pac), or hygromycin B phosphotransferase (Hph).

In some instances, at least one of the one or more selectable markers is a selectable marker for mammalian expression vectors. In some cases, at least one of the one or more selectable markers is adenine deaminase (ada), blasticidin S deaminases (Bsr, BSD), bleomycin-binding protein (Ble), Neomycin phosphotransferase (neo), histidinol dehydrogenase (hisD), glutamine synthetase (GS), dihydrofolate reductase (dhfr), cytosine deaminase (codA), puromycin N-acetyltransferase (Pac), or hygromycin B phosphotransferase (Hph).

In some embodiments, an isolated polynucleotide described herein comprises two or more selectable markers. In some instances, the first selectable marker is a selectable marker for mammalian expression vectors. In some cases, the first selectable marker is adenine deaminase (ada), blasticidin S deaminases (Bsr, BSD), bleomycin-binding protein (Ble), Neomycin phosphotransferase (neo), histidinol dehydrogenase (hisD), glutamine synthetase (GS), dihydrofolate reductase (dhfr), cytosine deaminase (codA), puromycin N-acetyltransferase (Pac), or hygromycin B phosphotransferase (Hph). In some instances, the second selectable marker is an antibiotic resistant gene. In some cases, the second selectable marker is ampicillin, chloramphenicol, kanamycin, tetracycline, polymyxin B, erythromycin, carbenicillin, streptomycin, spectinomycin, blasticidin S deaminases (Bsr, BSD), bleomycin-binding protein (Ble), Neomycin phosphotransferase (neo), puromycin N-acetyltransferase (Pac), zeocin (Sh bla), or hygromycin B phosphotransferase (Hph). In some cases, the second selectable marker is ampicillin, chloramphenicol, kanamycin, tetracycline, polymyxin B, erythromycin, bleomycin, carbenicillin, streptomycin, or spectinomycin. In some cases, the first selectable marker and the second selectable marker are different.

In some embodiments, the selectable marker is a gene involved in thymidylate synthase, thymidine kinase, dihydrofolate reductase, or glutamine synthetase. In some cases, one or more of the selectable markers comprise a gene involved in thymidylate synthase, thymidine kinase, dihydrofolate reductase, or glutamine synthetase.

In some embodiments, the selectable marker is a gene encoding a fluorescent protein. Exemplary fluorescent proteins include, but are not limited to:

Green fluorescent protein family members such as: green fluorescent protein (GFP), enhanced GFP (EGFP), Emerald, Superfolder GFP, Monomeric Azami Green, TagGFP2, mUKG, mWasabi, Clover, or mNeonGreen;

Blue fluorescent protein family members such as: TagBFP, mTagBFP2, Azurite, EBFP2, mKalama1, Sirius, Sapphire, or T-Sapphire;

Cyan fluorescent protein family members such as: enhanced cyan fluorescent protein (ECFP), Cerulean, SCFP3A, mTurquoise, mTurquoise2, monomeric Midoriishi-Cyan, TagCFP, or mTFP1;

Yellow fluorescent protein family members such as: enhanced yellow fluorescent protein (EYFP), Citrine, Venus, SYFP2, or TagYFP;

Orange fluorescent protein family members such as: monomeric Kusabira-Orange, mKOΛ, mKO2, mOrange, or mOrange2;

Red fluorescent protein family members such as: mRaspberry, mCherry, mStrawberry, mTangerine, tdTomato, TagRFP, TagRFP-T, mApple, mRuby, or mRuby2;

Far-Red fluorescent protein family members such as: mPlum, HcRed-Tandem, mKate2, mNeptune, or NirFP;

Near-IR protein family members such as: TagRFP657, IFP1.4, or iRFP;

Long Stokes Shift protein family members such as: mKeima Red, LSS-mKate1, LSS-mKate2, or mBeRFP;

Photoactivatible protein family members such as: PA-GFP, PAmCherry1, or PATagRFP;

Photoconvertible protein family members such as: Kaede (green), Kaede (red), KikGR1 (green), KikGR1 (red), PS-CFP2, mEos2 (green), mEos2 (red), mEos3.2 (green), mEos3.2 (red), or PSmOrange; and

Photoswitchable protein family members such as: Dronpa.

### Vectors

In certain embodiments, a vector described herein comprises a polynucleotide comprising an extended methylation-free CpG island encompassing dual divergently transcribed promoters. In some instances, the vector further comprises one or more promoters, enhancers, ribosome binding sites, RNA splice sites, polyadenylation sites, a replication origin, and/or transcriptional terminator sequences.

Promoters are specific nucleotide sequences in DNAs that allow initiation of transcription using DNAs as templates, and have a consensus sequence in general. In some instances, the promoters are constitutive promoters. In other instances, the promoters are inducible promoters. In additional instances, the promoters are specific promoters. In some cases, the promoters are eukaryotic promoters, or promoters used in a eukaryotic system. In other cases, the promoters are prokaryotic promoters, or promoters used in a prokaryotic system. In additional cases, the promoters are viral promoters, or promoters which are derived from a viral origin.

Exemplary eukaryotic promoters include, but are not limited to, CMV, EF1a, SV40, PGK1, Ubc, human beta actin, CAG, TRE, UAS, Ac5, polyhedron, CaMKIIa, GAL1-10, TEF1, GDS, ADH1, CaMV35S, Ubi, H1, and U6.

Exemplary prokaryotic promoters include, but are not limited to, T7, T7lac, Sp6, araBAD, trp, lac, Ptac, and pL.

Exemplary viral promoters include, but are not limited to, CaMV35S, SV40, CMV, and HSV TK promoter.

In some instances, a vector described herein comprises a constitutive promoter, an inducible promoter, or a specific promoter. In some cases, the vector comprises a eukaryotic promoter, a prokaryotic promoter, or a viral promoter. In some cases, the vector comprises a eukaryotic promoter selected from, for example, CMV, EF1a, SV40, PGK1, Ubc, human beta actin, CAG, TRE, UAS, Ac5, polyhedron, CaMKIIa, GAL1-10, TEF1, GDS, ADH1, CaMV35S, Ubi, H1, and U6; a prokaryotic promoter selected from, for example, T7, T7lac, Sp6, araBAD, trp, lac, Ptac, and pL; and/or a viral vector selected from, for example, CaMV35S, SV40, CMV, and HSV TK promoter.

Enhancers are nucleotide sequences that have the effect of enhancing promoter activity, and in general, often comprise about 100 bp. In some instances, enhancers augment transcription regardless of the orientation of their sequence. While enhancers themselves have no promoter activity, in some cases, they activate transcription from a distance of several kilo base pairs. Furthermore, enhancers are located optionally upstream or downstream of a gene region to be transcribed, and/or located within the gene, to activate the transcription.

Exemplary enhancers include, but are not limited to, WPRE; CMV enhancers; the R-U5′ segment in LTR of HTLV-I (Mol. Cell. Biol., Vol. 8(1), p. 466-472, 1988); SV40 enhancer; the intron sequence between exons 2 and 3 of rabbit β-globin (Proc. Natl. Acad. Sci. USA., Vol. 78(3), p. 1527-31, 1981); and the genome region of human growth hormone (J Immunol., Vol. 155(3), p. 1286-95, 1995).

In some embodiments, an isolated vector described herein comprises a mammalian vector, an insect vector, a yeast vector, or an algae vector. Mammalian vectors include, for example, transient expression vectors or stable expression vectors. Exemplary mammalian transient expression vectors include p3xFLAG-CMV 8, pFLAG-Myc-CMV 19, pFLAG-Myc-CMV 23, pFLAG-CMV 2, pFLAG-CMV 6a,b,c, pFLAG-CMV 5.1, pFLAG-CMV 5a,b,c, p3xFLAG-CMV 7.1, pFLAG-CMV 20, p3xFLAG-Myc-CMV 24, pCMV-FLAG-MAT1, pCMV-FLAG-MAT2, pBICEP-CMV 3, or pBICEP-CMV 4. Exemplary mammalian stable expression vectors include pFLAG-CMV 3, p3xFLAG-CMV 9, p3xFLAG-CMV 13, pFLAG-Myc-CMV 21, p3xFLAG-Myc-CMV 25, pFLAG-CMV 4, p3xFLAG-CMV 10, p3xFLAG-CMV 14, pFLAG-Myc-CMV 22, p3xFLAG-Myc-CMV 26, pBICEP-CMV 1, or pBICEP-CMV 2.

Insect vectors include, for example, pFastBac 1, pFastBac DUAL, pFastBac ET, pFastBac HTa, pFastBac HTb, pFastBac HTc, pFastBac M30a, pFastBact M30b, pFastBac, M30c, pVL1392, pVL1393, pVL1393 M10, pVL1393 M11, pVL1393 M12, FLAG vectors such as pPolh-FLAG1 or pPolh-MAT 2, or MAT vectors such as pPolh-MAT1, or pPolh-MAT2.

Yeast vectors include, for example, Gateway® pDEST™ 14 vector, Gateway® pDEST™ 15 vector, Gateway® pDEST™ 17 vector, Gateway® pDEST™ 24 vector, Gateway® pYES-DEST52 vector, pBAD-DEST49 Gateway® destination vector, pAO815 Pichia vector, pFLD1 Pichi pastoris vector, pGAPZA, B, & C Pichia pastoris vector, pPIC3.5K Pichia vector, pPIC6 A, B, & C Pichia vector, pPIC9K Pichia vector, pTEF1/Zeo, pYES2 yeast vector, pYES2/CT yeast vector, pYES2/NT A, B, & C yeast vector, or pYES3/CT yeast vector.

Algae vectors include, for example, pChlamy-4 vector or MCS vector.

Suitable protocols are readily known and/or available to those of skill in the art for delivery of a vector described herein to a host cell. Exemplary protocols include electroporation, calcium phosphate-mediated transfection, cell fusion, and those recommended by Invitrogen/Gibco for transfection of the CHO-S host cell-line. Generally, positive selection of cells containing the nucleic acid is achieved using agents such as, for example, hygromycin, G418, and puromycin. Following selection, the pool of resulting clones is, optionally, further subcloned to identify individual clones with the desired levels of protein expression.

In some instances, a vector described herein comprises at least 50%, 60%, 70%, 80%, 90%, 95%, or 99% sequence identity to a sequence selected from SEQ ID NOs: 2, 3, or 5. In some cases, the vector comprises at least 50% sequence identity to a sequence selected from SEQ ID NOs: 2, 3, or 5. In some cases, the vector comprises at least 60% sequence identity to a sequence selected from SEQ ID NOs: 2, 3, or 5. In some cases, the vector comprises at least 70% sequence identity to a sequence selected from SEQ ID NOs: 2, 3, or 5. In some cases, the vector comprises at least 80% sequence identity to a sequence selected from SEQ ID NOs: 2, 3, or 5. In some cases, the vector comprises at least 90% sequence identity to a sequence selected from SEQ ID NOs: 2, 3, or 5. In some cases, the vector comprises at least 95% sequence identity to a sequence selected from SEQ ID NOs: 2, 3, or 5. In some cases, the vector comprises at least 96% sequence identity to a sequence selected from SEQ ID NOs: 2, 3, or 5. In some cases, the vector comprises at least 97% sequence identity to a sequence selected from SEQ ID NOs: 2, 3, or 5. In some cases, the vector comprises at least 98% sequence identity to a sequence selected from SEQ ID NOs: 2, 3, or 5. In some cases, the vector comprises at least 99% sequence identity to a sequence selected from SEQ ID NOs: 2, 3, or 5. In some cases, the vector comprises 100% sequence identity to a sequence selected from SEQ ID NOs: 2, 3, or 5. In some cases, the vector consists of a sequence selected from SEQ ID NOs: 2, 3, or 5.

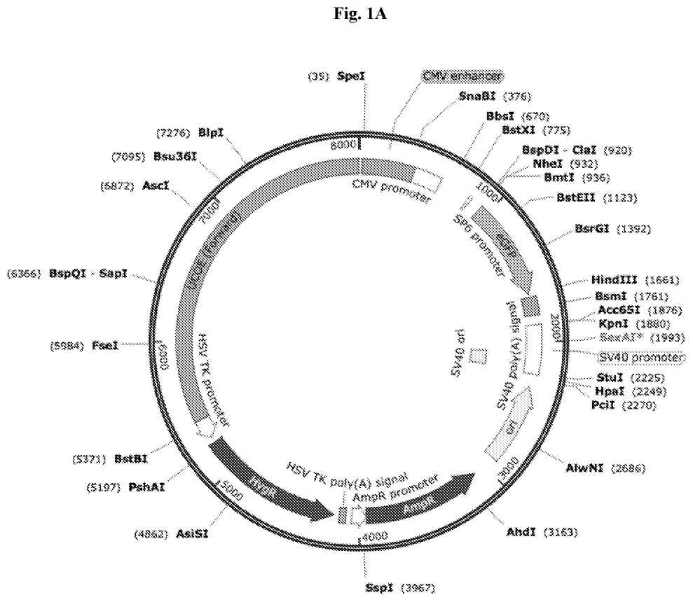

In some instances, a vector described herein is a vector illustrated in FIG. 1A, FIG. 1B, or FIG. 1C.

### Engineered Host Cells

In certain embodiments, a host cell described herein comprises a polynucleotide comprising an extended methylation-free CpG island encompassing dual divergently transcribed promoters or a vector comprising the polynucleotide comprising an extended methylation-free CpG island encompassing dual divergently transcribed promoters. Exemplary host cell systems include eukaryotic cell system, e.g., mammalian cell, insect cell, yeast cell, or plant cell. In some embodiments, the host cell is a mammalian host cell. In some cases, a mammalian host cell is a stable cell line, or a cell line that has incorporated a genetic material of interest into its own genome and has the capability to express the product of the genetic material after many generations of cell division. In other cases, a mammalian host cell is a transient cell line, or a cell line that has not incorporated a genetic material of interest into its own genome and does not have the capability to express the product of the genetic material after many generations of cell division.

Exemplary mammalian host cells include 293T cells, 293A cells, 293FT cells, 293F cells, 293 H cells, A549 cells, MDCK cells, CHO DG44 cells, CHO-S cells, CHO-K1 cells, Expi293F™ cells, Flp-In™ T-REx™ 293 cells, Flp-In™-293 cells, Flp-In™-3T3 cells, Flp-In™-BHK cells, Flp-In™-CHO cells, Flp-In™-CV-1 cells, Flp-In™-Jurkat cells, FreeStyle™ 293-F cells, FreeStyle™ CHO-S cells, GripTite™ 293 MSR cells, GS-CHO cells, HepaRG™ cells, T-REx™ Jurkat cells, Per.C6 cells, T-REx™-293 cells, T-REx™-CHO cells, and T-REx™-HeLa cells.

In some embodiments, the host cell is an insect host cell. Exemplary insect host cell include Drosophila S2 cells, Sf9 cells, Sf21 cells, High Five™ cells, and expresSF+® cells.

In some embodiments, the host cell is a yeast host cell. Exemplary yeast host cells include Pichia pastoris yeast strains such as GS115, KM71H, SMD1168, SMD1168H, and X-33; and Saccharomyces cerevisiae yeast strains such as INVSc1.

In some embodiments, the host cell is a plant host cell. In some instances, the plant cells comprise a cell from algae. Exemplary plant cell lines include strains from Chlamydomonas reinhardtii 137c, or Synechococcus elongatus PPC 7942.

### Methods of Enhancing the Expression of a Target Protein

In certain embodiments, described herein is a method of enhancing the expression of a target protein with a polynucleotide that comprises polynucleotide comprising an extended methylation-free CpG island encompassing dual divergently transcribed promoters. In some embodiments, the method comprises contacting a host cell with an isolated polynucleotide described above or an isolated vector described above, wherein the target gene of interest encodes the target protein; and culturing the host cell at a sufficient condition wherein the host cell expresses the target protein, thereby inducing an enhanced expression of the target protein.

In some cases, the host cell is a eukaryotic host cell. In some cases, the host cell comprises mammalian host cells such as 293T cells, 293A cells, 293FT cells, 293F cells, 293 H cells, A549 cells, MDCK cells, CHO DG44 cells, CHO-S cells, CHO-K1 cells, Expi293F™ cells, Flp-In™ T-REx™ 293 cells, Flp-In™-293 cells, Flp-In™-3T3 cells, Flp-In™-BHK cells, Flp-In™-CHO cells, Flp-In™-CV-1 cells, Flp-In™-Jurkat cells, FreeStyle™ 293-F cells, FreeStyle™ CHO-S cells, GripTite™ 293 MSR cells, GS-CHO cells, HepaRG™ cells, T-REx™ Jurkat cells, Per.C6 cells, T-REx™-293 cells, T-REx™-CHO cells, or T-REx™-HeLa cells.

In some cases, the host cell comprises insect host cell such as Drosophila S2 cells, Sf9 cells, Sf21 cells, High Five™ cells, or expresSF+® cells.

In other cases, the host cell comprises yeast host cells such as Pichia pastoris yeast strains including GS115, KM71H, SMD1168, SMD1168H, and X-33; and Saccharomyces cerevisiae yeast strains such as INVSc1.

In additional cases, the host cell comprises algae host cells from Chlamydomonas reinhardtii 137c or Synechococcus elongatus PPC 7942 strains.

In some instances, the sufficient condition is a suitable condition for culturing a particular host cell. For example, a suitable condition includes batch culture, fed-batch culture, continuous culture, or spin-tube culture. Suitable methods are known in the art and can be used to practice the present disclosure.

In some instances, cells are grown in any convenient volume chosen by the practitioner. For example, cells may be grown in small scale reaction vessels ranging in volume from a few milliliters to several liters. Alternatively, cells may be grown in large scale commercial Bioreactors ranging in volume from approximately at least 1 liter to 10, 50, 100, 250, 500, 1000, 2500, 5000, 8000, 10,000, 12,000, 15000, 20000 or 25000 liters or more, or any volume in between.

In some instances, the temperature of a cell culture is selected based primarily on the range of temperatures at which the cell culture remains viable and the range in which a high level of desired product (e.g., a recombinant protein) is produced. In general, most mammalian cells grow well and can produce desired products (e.g., recombinant proteins) from a range of about 25° C. to 42° C., although methods taught by the present disclosure are not limited to these temperatures. Certain mammalian cells grow well and can produce desired target protein from a range of about 35° C. to 40° C. In certain embodiments, a cell culture is grown at a temperature of about 20, 21, 22, 23, 24, 25, 26, 27, 28, 29, 30, 31, 32, 33, 34, 35, 36, 37, 38, 39, 40, 41, 42, 43, 44, or 45° C. at one or more times during the cell culture process. Those of ordinary skill in the art will be able to select appropriate temperature or temperatures in which to grow cells, depending on the particular needs of the cells and the particular production requirements of the practitioner. In some cases, the cells are grown for any amount of time, depending on the needs of the practitioner and the requirement of the cells themselves. In some embodiment, the cells are grown at 37° C. In some embodiments, the cells are grown at 36.5° C.

In some embodiments, host cells that contain and express a target polynucleotide sequence are identified by a variety of procedures known to those of skill in the art. These procedures include, but are not limited to, DNA-DNA or DNA-RNA hybridizations and protein bioassay or immunoassay techniques, which include, for example, membrane, solution, or chip based technologies for the detection and/or quantification of nucleic acid or protein.

A variety of protocols for detecting and measuring the expression of target polypeptide products, using either polyclonal or monoclonal antibodies specific for the product are known in the art. Examples include enzyme-linked immunosorbent assay (ELISA), radioimmunoassay (RIA), and fluorescence activated cell sorting (FACS). In some instances, a two-site, monoclonal-based immunoassay utilizing monoclonal antibodies reactive to two non-interfering epitopes on a given polypeptide is used. In other cases, a competitive binding assay is employed. These and other assays are described, among other places, in Hampton, R. et al. (1990, Serological Methods, a Laboratory Manual, APS Press, St Paul. Minn.) and Maddox, et al. (1983; J Exp. Med 158:1211-1216).

### Kits/Article of Manufacture

Disclosed herein, in certain embodiments, are kits and articles of manufacture for use with one or more polynucleotides, vectors, host cells, and methods described herein. Such kits include a carrier, package, or container that is compartmentalized to receive one or more containers such as vials, tubes, and the like, each of the container(s) comprising one of the separate elements to be used in a method described herein. Suitable containers include, for example, bottles, vials, and test tubes. In one embodiment, the containers are formed from a variety of materials such as glass or plastic.

The articles of manufacture provided herein contain packaging materials. Examples of pharmaceutical packaging materials include, but are not limited to, blister packs, bottles, tubes, bags, containers, bottles, and any packaging material suitable for a selected formulation and optionally intended mode of administration and treatment.

For example, the container(s) include a purified polypeptide described above or a purified vector described above. Such kits optionally include a plurality of host cells, an identifying description or label, and/or instructions relating to its use in the methods described herein.

A kit typically includes labels listing contents and/or instructions for use, and package inserts with instructions for use. A set of instructions will also typically be included.

In one embodiment, a label is on or associated with the container. In one embodiment, a label is on a container when letters, numbers or other characters forming the label are attached, molded or etched into the container itself; a label is associated with a container when it is present within a receptacle or carrier that also holds the container, e.g., as a package insert. In one embodiment, a label is used to indicate that the contents are to be used for a specific therapeutic application. The label also indicates directions for use of the contents, such as in the methods described herein.

### Certain Terminology

Unless defined otherwise, all technical and scientific terms used herein have the same meaning as is commonly understood by one of skill in the art to which the claimed subject matter belongs. It is to be understood that the foregoing general description and the following detailed description are exemplary and explanatory only and are not restrictive of any subject matter claimed. In this application, the use of the singular includes the plural unless specifically stated otherwise. It must be noted that, as used in the specification and the appended claims, the singular forms “a,” “an” and “the” include plural referents unless the context clearly dictates otherwise. In this application, the use of “or” means “and/or” unless stated otherwise. Furthermore, use of the term “including” as well as other forms, such as “include”, “includes,” and “included,” is not limiting.

As used herein, ranges and amounts can be expressed as “about” a particular value or range. About also includes the exact amount. Hence “about 5 μL” means “about 5 μL” and also “5 μL.” Generally, the term “about” includes an amount that would be expected to be within experimental error, e.g., within 5%, 10%, or 15%.

The section headings used herein are for organizational purposes only and are not to be construed as limiting the subject matter described.

An “island” corresponds to a single site, which in some cases is a single base position or a group of correlated base positions, e.g., a CpG island.

The term “proximal” means the end of the element in question that is closest to the reference element is close to or near the reference element. For example, a selectable marker that is located proximal to a polyadenylation signal can be more than about 50 bp, 100 bp, 200 bp, 500 bp, 1000 bp, 2000 bp, or 5000 bp away from the polyadenylation signal. In some cases, there are no other elements (e.g., promoters, enhancers, additional genes of interest) present between the element in question and the reference element. In other cases, an additional element (e.g., promoters, enhancers, additional genes of interest) is present between the element in question and the reference element.

The term “adjacent” means the end of the element in question that is closest to the reference element is next to the reference element. For example, a selectable marker that is located adjacent to a polyadenylation signal can be less than about 50 bp, 100 bp, 200 bp, 500 bp, 1000 bp, or 2000 bp away from the polyadenylation signal. In some cases, there are no other elements (e.g., promoters, enhancers, additional genes of interest) between the element in question and the reference element.

The term “operably linked” is intended to mean that the nucleotide sequence of interest is linked to the regulatory sequence(s) in a manner which allows for expression of the nucleotide sequence.

The term “regulatory sequence” is intended to include promoters, enhancers and other expression control elements (e.g., polyadenylation signals) described above. Such regulatory sequences are described, for example, in Goeddel, Gene Expression Technology: Methods in Enzymology 185, Academic Press, San Diego, Calif. (1990). Regulatory sequences include those which direct constitutive expression of a nucleotide sequence in many types of host cell and those which direct expression of the nucleotide sequence only in certain host cells (e.g., tissue-specific regulatory sequences). It will be appreciated by those skilled in the art that the design of the expression vector can depend on such factors as the choice of the host cell to be transformed, the level of expression of protein desired, and the like.

As used herein, the terms “transformation” and “transfection” are intended to refer to a variety of art-recognized techniques for introducing foreign nucleic acid into a host cell, including calcium phosphate or calcium chloride co-precipitation, DEAE-dextran-mediated transfection, lipofection, or electroporation.

### EXAMPLES

These examples are provided for illustrative purposes only and not to limit the scope of the claims provided herein.

### Example 1

CHO-GEnX² cells (a derivative of CHO-K1) were transfected with the following plasmids:

Plasmid 1 (SEQ ID NO: 4): GFP under control of the CMV enhancer/promoter

Plasmid 2 (SEQ ID NO: 5): GFP under control of the CMV enhancer/promoter with the 2591 bp UCOE sequence 5′ to the CMV enhancer (in the reverse orientation)

Plasmid 3 (also referred to as Vector 1) (SEQ ID NO: 2): GFP under control of the CMV enhancer/promoter with the 2591 bp UCOE sequence 5′ to the CMV enhancer (in the forward orientation)

Cells were plated in Transfectory CHO medium (Irvine Scientific) in 6 well plates at 2E6 cells/ml in a total volume of 2 ml. The cells were transfected with 2 μg of plasmid along with 0.2 μg of a 1.2 kb linear PCR fragment encoding the E. coli hygromycin B phosphotransferase gene using TransIT-PRO reagent as directed by the manufacturer (MirusBio, LLC).

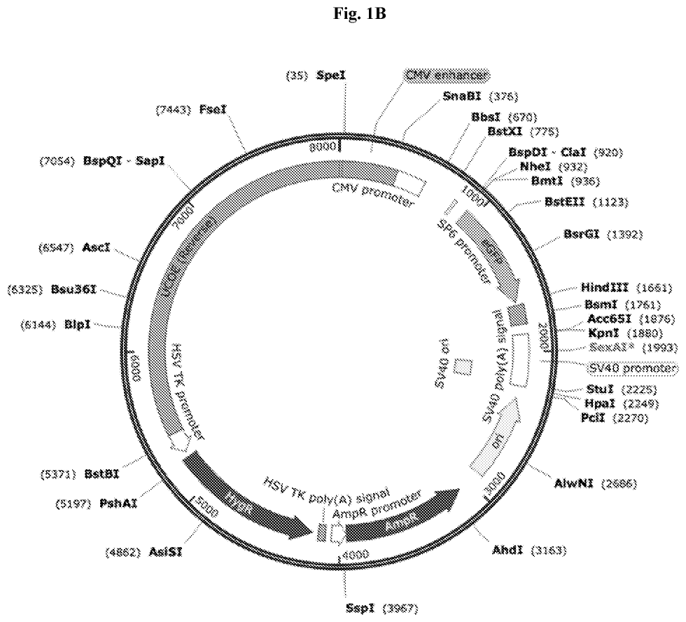

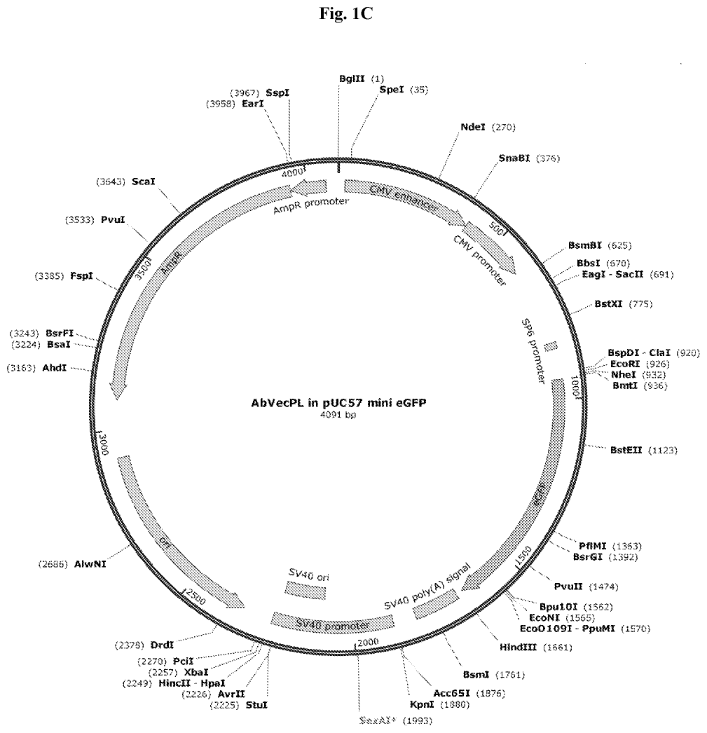

Cells were grown for one week post transfection at 37° C., 7% CO₂ and then transferred to shaker flasks in a final volume of 20 ml with Growth A medium (Irvine Scientific). Hygromycin was added to a final concentration of 300 μg/ml to select stable transfectants. The cells were grown in a shaking incubator at 37° C., 7% CO₂, 120 RPM until viable cell density reached roughly 1E6 cells/ml. The cells were then maintained in media containing 300 μg/ml hygromycin for one month at a total cell density not exceeding 2E6 cells/ml. This represented an additional 20-30 doublings. At this point (day 50 post-transfection) cells were analyzed for percent GFP-positivity using a Nexcelom Vision 5× Trio cell counter. Cells transfected with Plasmid 1, which does not contain the 2591 bp UCOE sequence, exhibited <0.1% GFP positivity (FIG. 2A and FIG. 2B). Cells transfected with Plasmid 2 and Plasmid 3 (or Vector 1) exhibited roughly 80% and 18% GFP positivity (respectively).

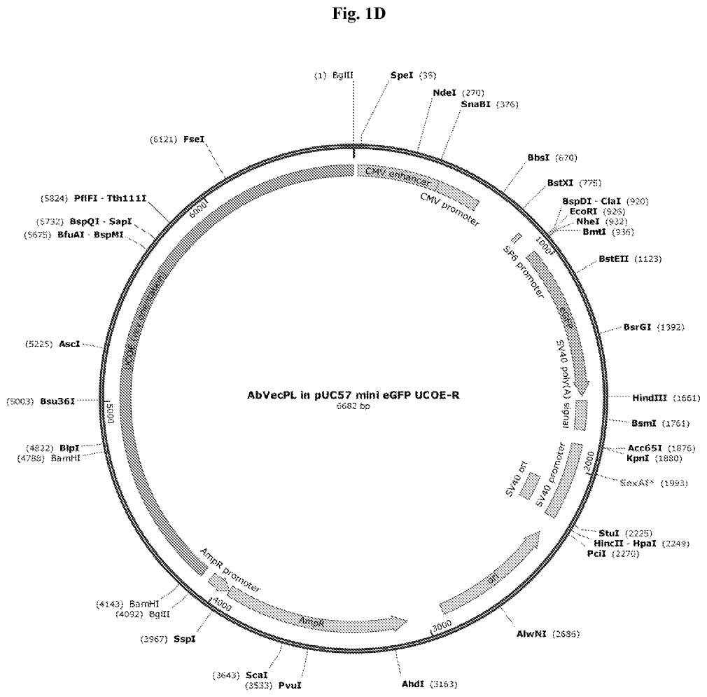

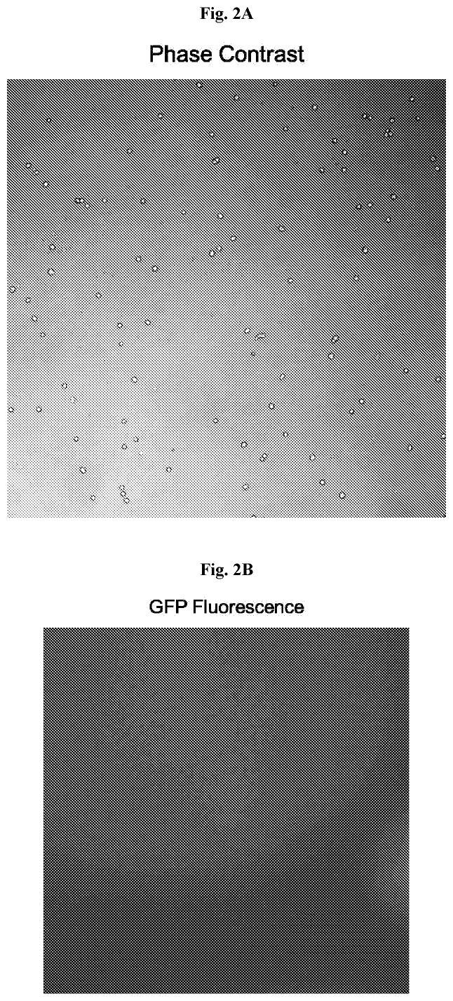

FIG. 3 shows illustrative phase contrast image (FIG. 3A) and GFP fluorescence (FIG. 3B) of cells transfected with Plasmid 2.

### Example 2

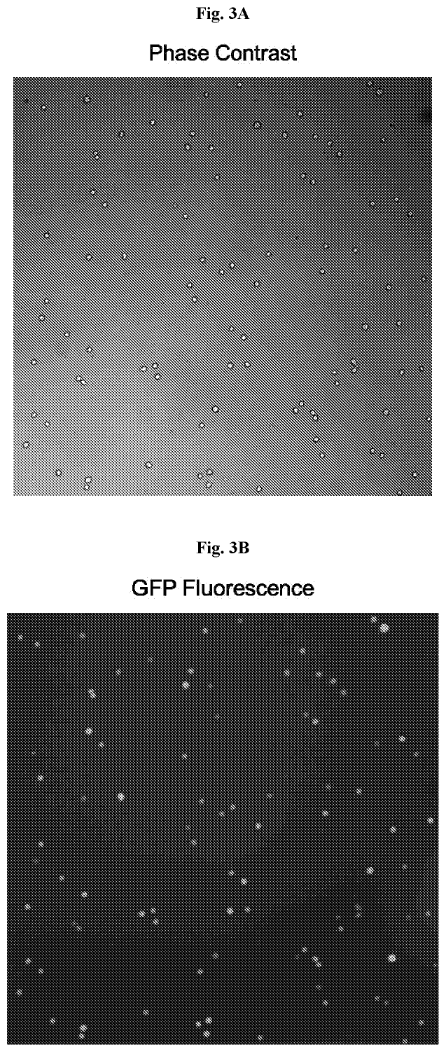

In a second set of experiments, HEK293 cells were transfected with Plasmid 3 (or Vector 1). In this experiment, cells were plated at 10000 cells/ml in 2 ml of RPMI in 6-well plates. About 2 ug of Plasmid 3 was transfected using TransIT Pro (MirusBio) as directed by the manufacturer. After 72 h, hygromycin was added at 50 ug/ml. After 7 days, the media was changed to remove the dead cells. After another 7 days, colonies were evident, and GFP fluorescence was observed (see FIG. 4).

### Example 3

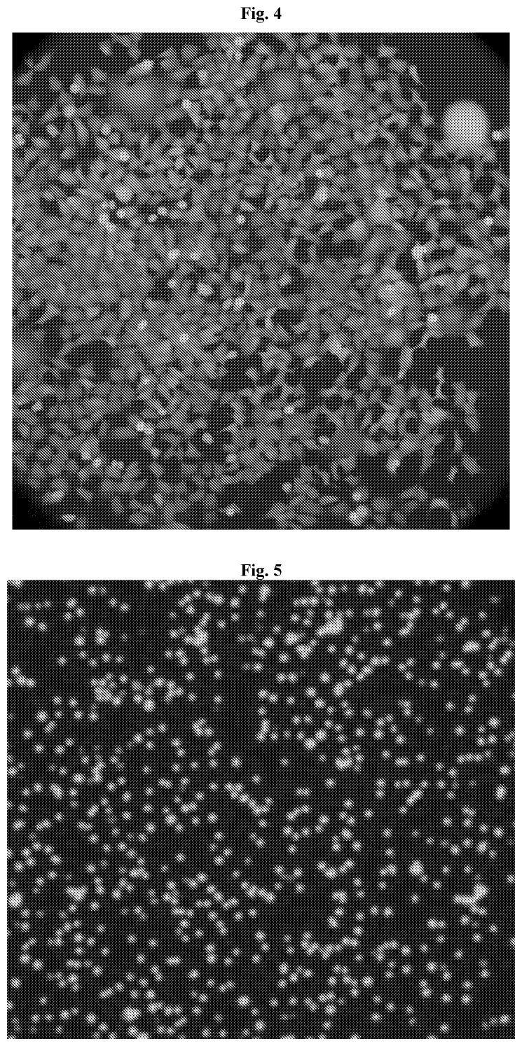

In a third experiment, GFP expression was monitored after a period of more than 3 months (see FIG. 5) and a loss of GFP expression was not observed in the cell culture. In brief, cells were transfected with a linear pIBS9 plasmid and selected with 200 μg/mL of Hygromycin on Day 4. The cell culture was then transferred to a 25 mL shaker on Day 14 and cultured in Hygromycin from Days 14-28, cultured without Hygromycin from Days 28-107, and measured GFP expression on Day 107.

Further, the culture was a stable pool and had been off selection for over 60 doublings. The mean fluorescence intensity (MFI) had increased by more than 20% since removal from selection.

### Example 4

TABLE 1
illustrates exemplary sequences disclosed herein.

| NAME | SEQUENCE | SEQ ID NO: |
|---|---|---|
| UCOE_1 | GAATTAGGAG CCAGCTCGGG GAAGTAAAAT GGTTTCTTTG | 1 |
| | CTGCAAGGCT GCCTTCAGCA GCAGGCTCAG GCCCGGATCG | |
| | CACTCCTCGC CCGGGGCCTG CGCCGGGAAC CCTTGCGGAG | |
| | GCCGCCGCCA CCCCCACCCG GCCGCGCACA CCGTGTGGCT | |
| | CGGGAGCGCA ATGGCGCCAT TTCGTCGAGG GGGAAGGAGG | |
| | GCGGAGCGCC TAAAACGAGG TGCGCAGGAC TCCGAGATCA | |
| | GAGCTCAAAA CACCCCAGAT CCCCCTCGGA ACGCCACCAA | |
| | AACGGGCCGG GGACAAAGGA AACCAGCTAA TGGCTCCCAC | |
| | GCGGGGGGGA AGGGGCCGAA CGGGCAGAGC CGCAGAGTGC | |
| | GGGACGCGAA AAACCGCTGC GAGCAAGGAG ACGGGAAAAT | |
| | GGCGGCCGCC AAATCCGGTT CCCGGGGGGG AGGAGGGAGG | |
| | GAGGGGGAGG GGAGCCGCGC AGAGCCGCTC CAGGGGGACC | |
| | CGGTGGCCGC CCGAACGAAC GCTCCGCCGC GCACACGCCC | |
| | CGGGCCCCGG GGCCCCGCGC ACGGCCCGCG GGGGAACCAG | |
| | GCCGGCCGGG CTCCCGCGCC CCTGCGGAGG ACGAGCCGCT | |
| | GGCCGCGGCC GGGCCCCATC CCCCACCCGG CTCAGGGAAA | |
| | TGGCGCCGGG CGGCTGAGGG CGGGGAAGGC GTTCGGACAA | |
| | TCGGGCCTCC GCCAACGACC CTCTCAGAGC CCCCGGGCCA | |
| | ACCGCCGCTC GGGCGGCCGC TCTCCCTCCC TCCGCCCGCC | |
| | CGCCCGCCGC GGCGGTGGCC GGCTTCCACG GCGGCCTCGC | |
| | CATGCCCTCG CCAACAACAA CTCATTGATT TCAAACCCGT | |
| | TACCTCCATC GCGGACTCAG TCGCTTCAGC CCGATTTCCC | |
| | GCAGCCGAGC GAGATGAGAA AGACCTCCGC GGACGCACAC | |
| | GCACGGGACT CGTCCTGACG CTGTGGGGGG GGAAGAGCAG | |
| | CCGCCGCTCG AGAAACCGCG CCGCGGGGGA AAGACCTGCA | |
| | CACAGGACCC GGCGCCGCTG CTACTGCCGC TGGAGCCGGG | |
| | ACCGCCGCTT TTCTAGAACC TTCCCCCCAC TAACGCGTCT | |
| | CCGCCTACGT CAGGCCGTTA CGCAAACGTC CTAACCGCCG | |
| | CCAATGGCGG GAAGGGCCTC GACACGCCTT TTGCGCCGGG | |
| | TGCGTGCGCC CTCCCCCTCC CGGGATAGCG AGGCCGCGTT | |
| | ACGTCACTTG CGCAACCGCG TTTGGTGAAT TGCGGAAGGC | |
| | TCGCGCGCCC AACGTGTTAC TACGTCCGGT GTGAGGTCCG | |
| | GCCCTCCCCC CCCACACCTC CGCGCAAGGA GCCGGAGAAA | |
| | CGGTCGCGCA GTTTGAAATT AACGCTGCCG GGCGGGGCTT | |
| | GCGTCGCCGC TCAGGCGTGA GGCCCTCGTC ACCCCGGCCG | |
| | CACTCCCTGC GGCGACGACG CCTTCGCCGA TCCTCGCTGC | |
| | GGGCCTCTCG AGGCGCGCCG GCCTTTGTTT GTTTGTGCCC | |
| | GTCCCCCAGG GGGGGTGCAG TCCGAGCGCG GCTGCCGCGG | |
| | CGGCACCCGA GGCCCTGGGA CCCCCGCGCC CCGTGAGAGG | |
| | CGGTGCTCAG AAGCTCCCCA GCCCCGCAGG CCCCGGGGCG | |
| | AGGAGGTGGG CGGAGCCCGG CTTTGGCGCG CTGGGCTTTG | |
| | ACGCAGAGCG GCCTCGGCGG TCCCGCCCGC CTCCCCTCAG | |
| | GAGACCCCCA AGGCGCCACG CCGCGGACGG TGAGTGGCGC | |
| | GCACGCCGCC CCCCGCAGGA CGCGTTCCCG GCGGGCCCCG | |
| | CGCGGCCTGG GCGTGGGCGG TGCCTCCCCT CCCCTCCCCC | |
| | CCGGCCGAGC CGCCCGCGCC CGCGCCCGCG CCCGCGCCCC | |
| | CTCCCCTCGG ATGTGGCTGA GCTGAGGCGC GCAGCCGGGA | |
| | GACTCTGCAG GATCCGCGCC CGGAGCATCT GAGCGGTGAA | |
| | GTCGGTGCCT TGCTTTTCTG GGTCGCGCGT GGCGCAGCGC | |
| | GGCGCGGCGC GGCGGGGCCG GGGATGCTCC GTGCGCCCGG | |
| | CTTCCCTGCG GGGAGGCTGC GGGCCCCCCG CGCCCCGCCG | |
| | TGGGCCCCCG GGACCCGGGG CTGCTGCGGG GAGGAGGCTT | |
| | GGCGGGGCCT GGCTGAGGCG CCGCCGCGCC GCGCCGCGCC | |
| | GCGCCGCGCC GCTGCACGGG GGAGGACGGC ACAGTCGTGC | |
| | CGAGGGTGGG AGAGCGATGA CAGCTGCGGG GCCTCCAGAC | |
| | AAAGCGCGCG GGTCCCCTCC CCCAGCCGCG CCGCGGGCCG | |
| | GCGGGCGCGG ATAACGTTAG AGCCGCCGCC GCCGCCGCGA | |
| | GGCCCCAGGC CCGAGCTGCA GCGCGCTCCC CGCTACGTGG | |
| | CGGCCGCCGG AGGGGGAGGC GTGCGCTGCT GGCGCTGCCC | |
| | TTGAGCCGCC GCCGCCTCCT TTGTTTCTCC CCGCGGAGGC | |
| | GCTGACCACG AGGCCCGCGC TCCCGAGTGG GGGGGGGCGG | |
| | GCTCCCAGCC GCGGCCTGTG ACCAGGCAAC GAGCGGGGAA | |
| | GGGGAGCGGG CGCGGAGCAC GGCGGTTCCT TCCAGCTTCA | |
| | TCCGTCCTCT GGCAGGATCC AGGCAGGCAG GCAGGCAGGA | |
| | GTATTCCTTC CCTAGGTGGA AAGCC | |
| Vector 1 | AGATCTATTC GAGCTCGCCC GACATTGATT ATTGACTAGT | 2 |
| | TATTAATAGT AATCAATTAC GGGGTCATTA GTTCATAGCC | |
| | CATATATGGA GTTCCGCGTT ACATAACTTA CGGTAAATGG | |
| | CCCGCCTGGC TGACCGCCCA ACGACCCCCG CCCATTGACG | |
| | TCAATAATGA CGTATGTTCC CATAGTAACG CCAATAGGGA | |
| | CTTTCCATTG ACGTCAATGG GTGGAGTATT TACGGTAAAC | |
| | TGCCCACTTG GCAGTACATC AAGTGTATCA TATGCCAAGT | |
| | ACGCCCCCTA TTGACGTCAA TGACGGTAAA TGGCCCGCCT | |
| | GGCATTATGC CCAGTACATG ACCTTATGGG ACTTTCCTAC | |
| | TTGGCAGTAC ATCTACGTAT TAGTCATCGC TATTACCATG | |
| | GTGATGCGGT TTTGGCAGTA CATCAATGGG CGTGGATAGC | |
| | GGTTTGACTC ACGGGGATTT CCAAGTCTCC ACCCCATTGA | |
| | CGTCAATGGG AGTTTGTTTT GGCACCAAAA TCAACGGGAC | |
| | TTTCCAAAAT GTCGTAACAA CTCCGCCCCA TTGACGCAAA | |
| | TGGGCGGTAG GCGTGTACGG TGGGAGGTCT ATATAAGCAG | |
| | AGCTCGTTTA GTGAACCGTC AGATCGCCTG GAGACGCCAT | |
| | CCACGCTGTT TTGACCTCCA TAGAAGACAC CGGGACCGAT | |
| | CCAGCCTCCG CGGCCGGGAA CGGTGCATTG GAACGCGGAT | |
| | TCCCCGTGCC AAGAGTGACG TAAGTACCGC CTATAGAGTC | |
| | TATAGGCCCA CCCCCTTGGC TTCGTTAGAA CGCGGCTACA | |
| | ATTAATACAT AACCTTGTGT ATCATACACA TACGATTTAG | |
| | GTGACACTAT AGAATAACAT CCACTTTGCC TTTCTCTCCA | |
| | CAGGTGTCCA CTCCCAGGTC CAACTGCACC TCGGTTCTAT | |
| | CGATTGAATT CGCTAGCGCC ACCATGTCTA AGGGAGAGGA | |
| | GCTGTTCACC GGAGTGGTGC CAATCCTGGT GGAGCTGGAC | |
| | GGCGATGTGA ATGGCCACAA GTTTTCTGTG AGCGGAGAGG | |
| | GAGAGGGCGA CGCAACCTAC GGCAAGCTGA CACTGAAGTT | |
| | CATCTGCACC ACAGGCAAGC TGCCCGTGCC TTGGCCAACC | |
| | CTGGTGACCA CATTCACATA CGGCGTGCAG TGTTTTTCCA | |
| | GGTATCCTGA CCACATGAAG CAGCACGATT TCTTTAAGTC | |
| | TGCCATGCCA GAGGGCTACG TGCAGGAGCG GACCATCTTC | |
| | TTTAAGGACG ATGGCAATTA TAAGACCAGA GCCGAGGTGA | |
| | AGTTCGAGGG CGACACACTG GTGAACAGGA TCGAGCTGAA | |
| | GGGCATCGAC TTTAAGGAGG ATGGCAATAT CCTGGGCCAC | |
| | AAGCTGGAGT ACAACTATAA TAGCCACAAC GTGTACATCA | |
| | TGGCCGATAA GCAGAAGAAC GGCATCAAGG CTAACTTCAA | |
| | GATCCGCCAC AACATCGAGG ACGGCTCCGT GCAGCTGGCC | |
| | GATCACTACC AGCAGAACAC CCCAATCGGC GACGGACCCG | |
| | TGCTGCTGCC TGATAATCAC TATCTGTCCA CACAGTCTGC | |
| | CCTGAGCAAG GACCCTAATG AGAAGCGGGA TCACATGGTG | |
| | CTGCTGGAGT TTGTGACCGC AGCAGGAATC ACACACGGAA | |
| | TGGACGAGCT GTATAAGTAA AAGCTTGGCC GCCATGGCCC | |
| | AACTTGTTTA TTGCAGCTTA TAATGGTTAC AAATAAAGCA | |
| | ATAGCATCAC AAATTTCACA AATAAAGCAT TTTTTTCACT | |
| | GCATTCTAGT TGTGGTTTGT CCAAACTCAT CAATGTATCT | |
| | TATCATGTCT GGATCGGGAA TTAATTCGGC GCAGCACCAT | |
| | GGCCTGAAAT AACCTCTGAA AGAGGAACTT GGTTAGGTAC | |
| | CTTCTGAGGC GGAAAGAACC ATCTGTGGAA TGTGTGTCAG | |
| | TTAGGGTGTG GAAAGTCCCC AGGCTCCCCA GCAGGCAGAA | |
| | GTATGCAAAG CATGCATCTC AATTAGTCAG CAACCAGGTG | |
| | TGGAAAGTCC CCAGGCTCCC CAGCAGGCAG AAGTATGCAA | |
| | AGCATGCATC TCAATTAGTC AGCAACCATA GTCCCGCCCC | |
| | TAACTCCGCC CATCCCGCCC CTAACTCCGC CCAGTTCCGC | |
| | CCATTCTCCG CCCCATGGCT GACTAATTTT TTTTATTTAT | |
| | GCAGAGGCCG AGGCCGCCTC GGCCTCTGAG CTATTCCAGA | |
| | AGTAGTGAGG AGGCTTTTTT GGAGGCCTAG GCTTTTGCAA | |
| | AAAGCTGTTA ACAGCTTCTA GAGGAAAGAA CATGTGAGCA | |
| | AAAGGCCAGC AAAAGGCCAG GAACCGTAAA AAGGCCGCGT | |
| | TGCTGGCGTT TTTCCATAGG CTCCGCCCCC CTGACGAGCA | |
| | TCACAAAAAT CGACGCTCAA GTCAGAGGTG GCGAAACCCG | |
| | ACAGGACTAT AAAGATACCA GGCGTTTCCC CCTGGAAGCT | |
| | CCCTCGTGCG CTCTCCTGTT CCGACCCTGC CGCTTACCGG | |
| | ATACCTGTCC GCCTTTCTCC CTTCGGGAAG CGTGGCGCTT | |
| | TCTCATAGCT CACGCTGTAG GTATCTCAGT TCGGTGTAGG | |
| | TCGTTCGCTC CAAGCTGGGC TGTGTGCACG AACCCCCCGT | |
| | TCAGCCCGAC CGCTGCGCCT TATCCGGTAA CTATCGTCTT | |
| | GAGTCCAACC CGGTAAGACA CGACTTATCG CCACTGGCAG | |
| | CAGCCACTGG TAACAGGATT AGCAGAGCGA GGTATGTAGG | |
| | CGGTGCTACA GAGTTCTTGA AGTGGTGGCC TAACTACGGC | |
| | TACACTAGAA GAACAGTATT TGGTATCTGC GCTCTGCTGA | |
| | AGCCAGTTAC CTTCGGAAAA AGAGTTGGTA GCTCTTGATC | |
| | CGGCAAACAA ACCACCGCTG GTAGCGGTGG TTTTTTTGTT | |
| | TGCAAGCAGC AGATTACGCG CAGAAAAAAA GGATCTCAAG | |
| | AAGATCCTTT GATCTTTTCT ACGGGGTCTG ACGCTCAGTG | |
| | GAACGAAAAC TCACGTTAAG GGATTTTGGT CATGAGATTA | |
| | TCAAAAAGGA TCTTCACCTA GATCCTTTTA AATTAAAAAT | |
| | GAAGTTTTAA ATCAATCTAA AGTATATATG AGTAAACTTG | |
| | GTCTGACAGT TACCAATGCT TAATCAGTGA GGCACCTATC | |
| | TCAGCGATCT GTCTATTTCG TTCATCCATA GTTGCCTGAC | |
| | TCCCCGTCGT GTAGATAACT ACGATACGGG AGGGCTTACC | |
| | ATCTGGCCCC AGTGCTGCAA TGATACCGCG AGACCCACGC | |
| | TCACCGGCTC CAGATTTATC AGCAATAAAC CAGCCAGCCG | |
| | GAAGGGCCGA GCGCAGAAGT GGTCCTGCAA CTTTATCCGC | |
| | CTCCATCCAG TCTATTAATT GTTGCCGGGA AGCTAGAGTA | |
| | AGTAGTTCGC CAGTTAATAG TTTGCGCAAC GTTGTTGCCA | |
| | TTGCTACAGG CATCGTGGTG TCACGCTCGT CGTTTGGTAT | |
| | GGCTTCATTC AGCTCCGGTT CCCAACGATC AAGGCGAGTT | |
| | ACATGATCCC CCATGTTGTG CAAAAAAGCG GTTAGCTCCT | |
| | TCGGTCCTCC GATCGTTGTC AGAAGTAAGT TGGCCGCAGT | |
| | GTTATCACTC ATGGTTATGG CAGCACTGCA TAATTCTCTT | |
| | ACTGTCATGC CATCCGTAAG ATGCTTTTCT GTGACTGGTG | |
| | AGTACTCAAC CAAGTCATTC TGAGAATAGT GTATGCGGCG | |
| | ACCGAGTTGC TCTTGCCCGG CGTCAATACG GGATAATACC | |
| | GCGCCACATA GCAGAACTTT AAAAGTGCTC ATCATTGGAA | |
| | AACGTTCTTC GGGGCGAAAA CTCTCAAGGA TCTTACCGCT | |
| | GTTGAGATCC AGTTCGATGT AACCCACTCG TGCACCCAAC | |
| | TGATCTTCAG CATCTTTTAC TTTCACCAGC GTTTCTGGGT | |
| | GAGCAAAAAC AGGAAGGCAA AATGCCGCAA AAAAGGGAAT | |
| | AAGGGCGACA CGGAAATGTT GAATACTCAT ACTCTTCCTT | |
| | TTTCAATATT ATTGAAGCAT TTATCAGGGT TATTGTCTCA | |
| | TGAGCGGATA CATATTTGAA TGTATTTAGA AAAATAAACA | |
| | AATAGGGGTT CCGCGCACAT TTCCCCGAAA AGTGCCACCT | |
| | GACGTCCCAA TAGATCCGAA CAAACGACCC AACACCCGTG | |
| | CGTTTTATTC TGTCTTTTTA TTGCCGTCAT AGCGCGGGTT | |
| | CCTTCCGGTA TTGTCTCCTT CCGTGTTTCA GTTAGCCTCC | |
| | CCCATCTCCC GATCCGGACG AGTGCTGGGG CGTCGGTTTC | |
| | CACTATCGGC GAGTACTTCT ACACAGCCAT CGGTCCAGAC | |
| | GGCCGCGCTT CTGCGGGCGA TTTGTGTACG CCCGACAGTC | |
| | CCGGCTCCGG ATCGGACGAT TGCGTCGCAT CGACCCTGCG | |
| | CCCAAGCTGC ATCATCGAAA TTGCCGTCAA CCAAGCTCTG | |
| | ATAGAGTTGG TCAAGACCAA TGCGGAGCAT ATACGCCCGG | |
| | AGCCGCGGCG ATCCTGCAAG CTCCGGATGC CTCCGCTCGA | |
| | AGTAGCGCGT CTGCTGCTCC ATACAAGCCA ACCACGGCCT | |
| | CCAGAAGAAG ATGTTGGCGA CCTCGTATTG GGAATCCCCG | |
| | AACATCGCCT CGCTCCAGTC AATGACCGCT GTTATGCGGC | |
| | CATTGTCCGT CAGGACATTG TTGGAGCCGA AATCCGCGTG | |
| | CACGAGGTGC CGGACTTCGG GGCAGTCCTC GGCCCAAAGC | |
| | ATCAGCTCAT CGAGAGCCTG CGCGACGGAC GCACTGACGG | |
| | TGTCGTCCAT CACAGTTTGC CAGTGATACA CATGGGGATC | |
| | AGCAATCGCG CATATGAAAT CACGCCATGT AGTGTATTGA | |
| | CCGATTCCTT GCGGTCCGAA TGGGCCGAAC CCGCTCGTCT | |
| | GGCTAAGATC GGCCGCAGCG ATCGCATCCA TGGCCTCCGC | |
| | GACCGGCTGC AGAACAGCGG GCAGTTCGGT TTCAGGCAGG | |
| | TCTTGCAACG TGACACCCTG TGCACGGCGG GAGATGCAAT | |
| | AGGTCAGGCT CTCGCTGAAT TCCCCAATGT CAAGCACTTC | |
| | CGGAATCGGG AGCGCGGCCG ATGCAAAGTG CCGATAAACA | |
| | TAACGATCTT TGTAGAAACC ATCGGCGCAG CTATTTACCC | |
| | GCAGGACATA TCCACGCCCT CCTACATCGA AGCTGAAAGC | |
| | ACGAGATTCT TCGCCCTCCG AGAGCTGCAT CAGGTCGGAG | |
| | ACGCTGTCGA ACTTTTCGAT CAGAAACTTC TCGACAGACG | |
| | TCGCGGTGAG TTCAGGCTTT TTCATATCTC ATTGCCCCCC | |
| | GGGATCTGCG GCACGCTGTT GACGCTGTTA AGCGGGTCGC | |
| | TGCAGGGTCG CTCGGTGTTC GAGGCCACAC GCGTCACCTT | |
| | AATATGCGAA GTGGACCTCG GACCGCGCCG CCCCGACTGC | |
| | ATCTGCGTGT TCGAATTCGC CAATGACAAG ACGCTGGGCG | |
| | GGGTTTGTGT CATAGATCTG AATTAGGAGC CAGCTCGGGG | |
| | AAGTAAAATG GTTTCTTTGC TGCAAGGCTG CCTTCAGCAG | |
| | CAGGCTCAGG CCCGGATCGC ACTCCTCGCC CGGGGCCTGC | |
| | GCCGGGAACC CTTGCGGAGG CCGCCGCCAC CCCCACCCGG | |
| | CCGCGCACAC CGTGTGGCTC GGGAGCGCAA TGGCGCCATT | |
| | TCGTCGAGGG GGAAGGAGGG CGGAGCGCCT AAAACGAGGT | |
| | GCGCAGGACT CCGAGATCAG AGCTCAAAAC ACCCCAGATC | |
| | CCCCTCGGAA CGCCACCAAA ACGGGCCGGG GACAAAGGAA | |
| | ACCAGCTAAT GGCTCCCACG CGGGGGGGAA GGGGCCGAAC | |
| | GGGCAGAGCC GCAGAGTGCG GGACGCGAAA AACCGCTGCG | |
| | AGCAAGGAGA CGGGAAAATG GCGGCCGCCA AATCCGGTTC | |
| | CCGGGGGGGA GGAGGGAGGG AGGGGGAGGG GAGCCGCGCA | |
| | GAGCCGCTCC AGGGGGACCC GGTGGCCGCC CGAACGAACG | |
| | CTCCGCCGCG CACACGCCCC GGGCCCCGGG GCCCCGCGCA | |
| | CGGCCCGCGG GGGAACCAGG CCGGCCGGGC TCCCGCGCCC | |
| | CTGCGGAGGA CGAGCCGCTG GCCGCGGCCG GGCCCCATCC | |
| | CCCACCCGGC TCAGGGAAAT GGCGCCGGGC GGCTGAGGGC | |
| | GGGGAAGGCG TTCGGACAAT CGGGCCTCCG CCAACGACCC | |
| | TCTCAGAGCC CCCGGGCCAA CCGCCGCTCG GGCGGCCGCT | |
| | CTCCCTCCCT CCGCCCGCCC GCCCGCCGCG GCGGTGGCCG | |
| | GCTTCCACGG CGGCCTCGCC ATGCCCTCGC CAACAACAAC | |
| | TCATTGATTT CAAACCCGTT ACCTCCATCG CGGACTCAGT | |
| | CGCTTCAGCC CGATTTCCCG CAGCCGAGCG AGATGAGAAA | |
| | GACCTCCGCG GACGCACACG CACGGGACTC GTCCTGACGC | |
| | TGTGGGGGGG GAAGAGCAGC CGCCGCTCGA GAAACCGCGC | |
| | CGCGGGGGAA AGACCTGCAC ACAGGACCCG GCGCCGCTGC | |
| | TACTGCCGCT GGAGCCGGGA CCGCCGCTTT TCTAGAACCT | |
| | TCCCCCCACT AACGCGTCTC CGCCTACGTC AGGCCGTTAC | |
| | GCAAACGTCC TAACCGCCGC CAATGGCGGG AAGGGCCTCG | |
| | ACACGCCTTT TGCGCCGGGT GCGTGCGCCC TCCCCCTCCC | |
| | GGGATAGCGA GGCCGCGTTA CGTCACTTGC GCAACCGCGT | |
| | TTGGTGAATT GCGGAAGGCT CGCGCGCCCA ACGTGTTACT | |
| | ACGTCCGGTG TGAGGTCCGG CCCTCCCCCC CCACACCTCC | |
| | GCGCAAGGAG CCGGAGAAAC GGTCGCGCAG TTTGAAATTA | |
| | ACGCTGCCGG GCGGGGCTTG CGTCGCCGCT CAGGCGTGAG | |
| | GCCCTCGTCA CCCCGGCCGC ACTCCCTGCG GCGACGACGC | |
| | CTTCGCCGAT CCTCGCTGCG GGCCTCTCGA GGCGCGCCGG | |
| | CCTTTGTTTG TTTGTGCCCG TCCCCCAGGG GGGGTGCAGT | |
| | CCGAGCGCGG CTGCCGCGGC GGCACCCGAG GCCCTGGGAC | |
| | CCCCGCGCCC CGTGAGAGGC GGTGCTCAGA AGCTCCCCAG | |
| | CCCCGCAGGC CCCGGGGCGA GGAGGTGGGC GGAGCCCGGC | |
| | TTTGGCGCGC TGGGCTTTGA CGCAGAGCGG CCTCGGCGGT | |
| | CCCGCCCGCC TCCCCTCAGG AGACCCCCAA GGCGCCACGC | |
| | CGCGGACGGT GAGTGGCGCG CACGCCGCCC CCCGCAGGAC | |
| | GCGTTCCCGG CGGGCCCCGC GCGGCCTGGG CGTGGGCGGT | |
| | GCCTCCCCTC CCCTCCCCCC CGGCCGAGCC GCCCGCGCCC | |
| | GCGCCCGCGC CCGCGCCCCC TCCCCTCGGA TGTGGCTGAG | |
| | CTGAGGCGCG CAGCCGGGAG ACTCTGCAGG ATCCGCGCCC | |
| | GGAGCATCTG AGCGGTGAAG TCGGTGCCTT GCTTTTCTGG | |
| | GTCGCGCGTG GCGCAGCGCG GCGCGGCGCG GCGGGGCCGG | |
| | GGATGCTCCG TGCGCCCGGC TTCCCTGCGG GGAGGCTGCG | |
| | GGCCCCCCGC GCCCCGCCGT GGGCCCCCGG GACCCGGGGC | |
| | TGCTGCGGGG AGGAGGCTTG GCGGGGCCTG GCTGAGGCGC | |
| | CGCCGCGCCG CGCCGCGCCG CGCCGCGCCG CTGCACGGGG | |
| | GAGGACGGCA CAGTCGTGCC GAGGGTGGGA GAGCGATGAC | |
| | AGCTGCGGGG CCTCCAGACA AAGCGCGCGG GTCCCCTCCC | |
| | CCAGCCGCGC CGCGGGCCGG CGGGCGCGGA TAACGTTAGA | |
| | GCCGCCGCCG CCGCCGCGAG GCCCCAGGCC CGAGCTGCAG | |
| | CGCGCTCCCC GCTACGTGGC GGCCGCCGGA GGGGGAGGCG | |
| | TGCGCTGCTG GCGCTGCCCT TGAGCCGCCG CCGCCTCCTT | |
| | TGTTTCTCCC CGCGGAGGCG CTGACCACGA GGCCCGCGCT | |
| | CCCGAGTGGG GGGGGGCGGG CTCCCAGCCG CGGCCTGTGA | |
| | CCAGGCAACG AGCGGGGAAG GGGAGCGGGC GCGGAGCACG | |
| | GCGGTTCCTT CCAGCTTCAT CCGTCCTCTG GCAGGATCCA | |
| | GGCAGGCAGG CAGGCAGGAG TATTCCTTCC CTAGGTGGAA | |
| | AGCC | |
| Vector 2 | AGATCTATTC GAGCTCGCCC GACATTGATT ATTGACTAGT | 3 |
| | TATTAATAGT AATCAATTAC GGGGTCATTA GTTCATAGCC | |
| | CATATATGGA GTTCCGCGTT ACATAACTTA CGGTAAATGG | |
| | CCCGCCTGGC TGACCGCCCA ACGACCCCCG CCCATTGACG | |
| | TCAATAATGA CGTATGTTCC CATAGTAACG CCAATAGGGA | |
| | CTTTCCATTG ACGTCAATGG GTGGAGTATT TACGGTAAAC | |
| | TGCCCACTTG GCAGTACATC AAGTGTATCA TATGCCAAGT | |
| | ACGCCCCCTA TTGACGTCAA TGACGGTAAA TGGCCCGCCT | |
| | GGCATTATGC CCAGTACATG ACCTTATGGG ACTTTCCTAC | |
| | TTGGCAGTAC ATCTACGTAT TAGTCATCGC TATTACCATG | |
| | GTGATGCGGT TTTGGCAGTA CATCAATGGG CGTGGATAGC | |
| | GGTTTGACTC ACGGGGATTT CCAAGTCTCC ACCCCATTGA | |
| | CGTCAATGGG AGTTTGTTTT GGCACCAAAA TCAACGGGAC | |
| | TTTCCAAAAT GTCGTAACAA CTCCGCCCCA TTGACGCAAA | |
| | TGGGCGGTAG GCGTGTACGG TGGGAGGTCT ATATAAGCAG | |
| | AGCTCGTTTA GTGAACCGTC AGATCGCCTG GAGACGCCAT | |
| | CCACGCTGTT TTGACCTCCA TAGAAGACAC CGGGACCGAT | |
| | CCAGCCTCCG CGGCCGGGAA CGGTGCATTG GAACGCGGAT | |
| | TCCCCGTGCC AAGAGTGACG TAAGTACCGC CTATAGAGTC | |
| | TATAGGCCCA CCCCCTTGGC TTCGTTAGAA CGCGGCTACA | |
| | ATTAATACAT AACCTTGTGT ATCATACACA TACGATTTAG | |
| | GTGACACTAT AGAATAACAT CCACTTTGCC TTTCTCTCCA | |
| | CAGGTGTCCA CTCCCAGGTC CAACTGCACC TCGGTTCTAT | |
| | CGATTGAATT CGCTAGCGCC ACCATGTCTA AGGGAGAGGA | |
| | GCTGTTCACC GGAGTGGTGC CAATCCTGGT GGAGCTGGAC | |
| | GGCGATGTGA ATGGCCACAA GTTTTCTGTG AGCGGAGAGG | |
| | GAGAGGGCGA CGCAACCTAC GGCAAGCTGA CACTGAAGTT | |
| | CATCTGCACC ACAGGCAAGC TGCCCGTGCC TTGGCCAACC | |
| | CTGGTGACCA CATTCACATA CGGCGTGCAG TGTTTTTCCA | |
| | GGTATCCTGA CCACATGAAG CAGCACGATT TCTTTAAGTC | |
| | TGCCATGCCA GAGGGCTACG TGCAGGAGCG GACCATCTTC | |
| | TTTAAGGACG ATGGCAATTA TAAGACCAGA GCCGAGGTGA | |
| | AGTTCGAGGG CGACACACTG GTGAACAGGA TCGAGCTGAA | |
| | GGGCATCGAC TTTAAGGAGG ATGGCAATAT CCTGGGCCAC | |
| | AAGCTGGAGT ACAACTATAA TAGCCACAAC GTGTACATCA | |
| | TGGCCGATAA GCAGAAGAAC GGCATCAAGG CTAACTTCAA | |
| | GATCCGCCAC AACATCGAGG ACGGCTCCGT GCAGCTGGCC | |
| | GATCACTACC AGCAGAACAC CCCAATCGGC GACGGACCCG | |
| | TGCTGCTGCC TGATAATCAC TATCTGTCCA CACAGTCTGC | |
| | CCTGAGCAAG GACCCTAATG AGAAGCGGGA TCACATGGTG | |
| | CTGCTGGAGT TTGTGACCGC AGCAGGAATC ACACACGGAA | |
| | TGGACGAGCT GTATAAGTAA AAGCTTGGCC GCCATGGCCC | |
| | AACTTGTTTA TTGCAGCTTA TAATGGTTAC AAATAAAGCA | |
| | ATAGCATCAC AAATTTCACA AATAAAGCAT TTTTTTCACT | |
| | GCATTCTAGT TGTGGTTTGT CCAAACTCAT CAATGTATCT | |
| | TATCATGTCT GGATCGGGAA TTAATTCGGC GCAGCACCAT | |
| | GGCCTGAAAT AACCTCTGAA AGAGGAACTT GGTTAGGTAC | |
| | CTTCTGAGGC GGAAAGAACC ATCTGTGGAA TGTGTGTCAG | |
| | TTAGGGTGTG GAAAGTCCCC AGGCTCCCCA GCAGGCAGAA | |
| | GTATGCAAAG CATGCATCTC AATTAGTCAG CAACCAGGTG | |
| | TGGAAAGTCC CCAGGCTCCC CAGCAGGCAG AAGTATGCAA | |
| | AGCATGCATC TCAATTAGTC AGCAACCATA GTCCCGCCCC | |
| | TAACTCCGCC CATCCCGCCC CTAACTCCGC CCAGTTCCGC | |
| | CCATTCTCCG CCCCATGGCT GACTAATTTT TTTTATTTAT | |
| | GCAGAGGCCG AGGCCGCCTC GGCCTCTGAG CTATTCCAGA | |
| | AGTAGTGAGG AGGCTTTTTT GGAGGCCTAG GCTTTTGCAA | |
| | AAAGCTGTTA ACAGCTTCTA GAGGAAAGAA CATGTGAGCA | |
| | AAAGGCCAGC AAAAGGCCAG GAACCGTAAA AAGGCCGCGT | |
| | TGCTGGCGTT TTTCCATAGG CTCCGCCCCC CTGACGAGCA | |
| | TCACAAAAAT CGACGCTCAA GTCAGAGGTG GCGAAACCCG | |
| | ACAGGACTAT AAAGATACCA GGCGTTTCCC CCTGGAAGCT | |
| | CCCTCGTGCG CTCTCCTGTT CCGACCCTGC CGCTTACCGG | |
| | ATACCTGTCC GCCTTTCTCC CTTCGGGAAG CGTGGCGCTT | |
| | TCTCATAGCT CACGCTGTAG GTATCTCAGT TCGGTGTAGG | |
| | TCGTTCGCTC CAAGCTGGGC TGTGTGCACG AACCCCCCGT | |
| | TCAGCCCGAC CGCTGCGCCT TATCCGGTAA CTATCGTCTT | |
| | GAGTCCAACC CGGTAAGACA CGACTTATCG CCACTGGCAG | |
| | CAGCCACTGG TAACAGGATT AGCAGAGCGA GGTATGTAGG | |
| | CGGTGCTACA GAGTTCTTGA AGTGGTGGCC TAACTACGGC | |
| | TACACTAGAA GAACAGTATT TGGTATCTGC GCTCTGCTGA | |
| | AGCCAGTTAC CTTCGGAAAA AGAGTTGGTA GCTCTTGATC | |
| | CGGCAAACAA ACCACCGCTG GTAGCGGTGG TTTTTTTGTT | |
| | TGCAAGCAGC AGATTACGCG CAGAAAAAAA GGATCTCAAG | |
| | AAGATCCTTT GATCTTTTCT ACGGGGTCTG ACGCTCAGTG | |
| | GAACGAAAAC TCACGTTAAG GGATTTTGGT CATGAGATTA | |
| | TCAAAAAGGA TCTTCACCTA GATCCTTTTA AATTAAAAAT | |
| | GAAGTTTTAA ATCAATCTAA AGTATATATG AGTAAACTTG | |
| | GTCTGACAGT TACCAATGCT TAATCAGTGA GGCACCTATC | |
| | TCAGCGATCT GTCTATTTCG TTCATCCATA GTTGCCTGAC | |
| | TCCCCGTCGT GTAGATAACT ACGATACGGG AGGGCTTACC | |
| | ATCTGGCCCC AGTGCTGCAA TGATACCGCG AGACCCACGC | |
| | TCACCGGCTC CAGATTTATC AGCAATAAAC CAGCCAGCCG | |
| | GAAGGGCCGA GCGCAGAAGT GGTCCTGCAA CTTTATCCGC | |
| | CTCCATCCAG TCTATTAATT GTTGCCGGGA AGCTAGAGTA | |
| | AGTAGTTCGC CAGTTAATAG TTTGCGCAAC GTTGTTGCCA | |
| | TTGCTACAGG CATCGTGGTG TCACGCTCGT CGTTTGGTAT | |
| | GGCTTCATTC AGCTCCGGTT CCCAACGATC AAGGCGAGTT | |
| | ACATGATCCC CCATGTTGTG CAAAAAAGCG GTTAGCTCCT | |
| | TCGGTCCTCC GATCGTTGTC AGAAGTAAGT TGGCCGCAGT | |
| | GTTATCACTC ATGGTTATGG CAGCACTGCA TAATTCTCTT | |
| | ACTGTCATGC CATCCGTAAG ATGCTTTTCT GTGACTGGTG | |
| | AGTACTCAAC CAAGTCATTC TGAGAATAGT GTATGCGGCG | |
| | ACCGAGTTGC TCTTGCCCGG CGTCAATACG GGATAATACC | |
| | GCGCCACATA GCAGAACTTT AAAAGTGCTC ATCATTGGAA | |
| | AACGTTCTTC GGGGCGAAAA CTCTCAAGGA TCTTACCGCT | |
| | GTTGAGATCC AGTTCGATGT AACCCACTCG TGCACCCAAC | |
| | TGATCTTCAG CATCTTTTAC TTTCACCAGC GTTTCTGGGT | |
| | GAGCAAAAAC AGGAAGGCAA AATGCCGCAA AAAAGGGAAT | |
| | AAGGGCGACA CGGAAATGTT GAATACTCAT ACTCTTCCTT | |
| | TTTCAATATT ATTGAAGCAT TTATCAGGGT TATTGTCTCA | |
| | TGAGCGGATA CATATTTGAA TGTATTTAGA AAAATAAACA | |
| | AATAGGGGTT CCGCGCACAT TTCCCCGAAA AGTGCCACCT | |
| | GACGTCCCAA TAGATCCGAA CAAACGACCC AACACCCGTG | |
| | CGTTTTATTC TGTCTTTTTA TTGCCGTCAT AGCGCGGGTT | |
| | CCTTCCGGTA TTGTCTCCTT CCGTGTTTCA GTTAGCCTCC | |
| | CCCATCTCCC GATCCGGACG AGTGCTGGGG CGTCGGTTTC | |
| | CACTATCGGC GAGTACTTCT ACACAGCCAT CGGTCCAGAC | |
| | GGCCGCGCTT CTGCGGGCGA TTTGTGTACG CCCGACAGTC | |
| | CCGGCTCCGG ATCGGACGAT TGCGTCGCAT CGACCCTGCG | |
| | CCCAAGCTGC ATCATCGAAA TTGCCGTCAA CCAAGCTCTG | |
| | ATAGAGTTGG TCAAGACCAA TGCGGAGCAT ATACGCCCGG | |
| | AGCCGCGGCG ATCCTGCAAG CTCCGGATGC CTCCGCTCGA | |
| | AGTAGCGCGT CTGCTGCTCC ATACAAGCCA ACCACGGCCT | |
| | CCAGAAGAAG ATGTTGGCGA CCTCGTATTG GGAATCCCCG | |
| | AACATCGCCT CGCTCCAGTC AATGACCGCT GTTATGCGGC | |
| | CATTGTCCGT CAGGACATTG TTGGAGCCGA AATCCGCGTG | |
| | CACGAGGTGC CGGACTTCGG GGCAGTCCTC GGCCCAAAGC | |
| | ATCAGCTCAT CGAGAGCCTG CGCGACGGAC GCACTGACGG | |
| | TGTCGTCCAT CACAGTTTGC CAGTGATACA CATGGGGATC | |
| | AGCAATCGCG CATATGAAAT CACGCCATGT AGTGTATTGA | |
| | CCGATTCCTT GCGGTCCGAA TGGGCCGAAC CCGCTCGTCT | |
| | GGCTAAGATC GGCCGCAGCG ATCGCATCCA TGGCCTCCGC | |
| | GACCGGCTGC AGAACAGCGG GCAGTTCGGT TTCAGGCAGG | |
| | TCTTGCAACG TGACACCCTG TGCACGGCGG GAGATGCAAT | |
| | AGGTCAGGCT CTCGCTGAAT TCCCCAATGT CAAGCACTTC | |
| | CGGAATCGGG AGCGCGGCCG ATGCAAAGTG CCGATAAACA | |
| | TAACGATCTT TGTAGAAACC ATCGGCGCAG CTATTTACCC | |
| | GCAGGACATA TCCACGCCCT CCTACATCGA AGCTGAAAGC | |
| | ACGAGATTCT TCGCCCTCCG AGAGCTGCAT CAGGTCGGAG | |
| | ACGCTGTCGA ACTTTTCGAT CAGAAACTTC TCGACAGACG | |
| | TCGCGGTGAG TTCAGGCTTT TTCATATCTC ATTGCCCCCC | |
| | GGGATCTGCG GCACGCTGTT GACGCTGTTA AGCGGGTCGC | |
| | TGCAGGGTCG CTCGGTGTTC GAGGCCACAC GCGTCACCTT | |
| | AATATGCGAA GTGGACCTCG GACCGCGCCG CCCCGACTGC | |
| | ATCTGCGTGT TCGAATTCGC CAATGACAAG ACGCTGGGCG | |
| | GGGTTTGTGT CATAGATCTG GCTTTCCACC TAGGGAAGGA | |
| | ATACTCCTGC CTGCCTGCCT GCCTGGATCC TGCCAGAGGA | |
| | CGGATGAAGC TGGAAGGAAC CGCCGTGCTC CGCGCCCGCT | |
| | CCCCTTCCCC GCTCGTTGCC TGGTCACAGG CCGCGGCTGG | |
| | GAGCCCGCCC CCCCCCACTC GGGAGCGCGG GCCTCGTGGT | |
| | CAGCGCCTCC GCGGGGAGAA ACAAAGGAGG CGGCGGCGGC | |
| | TCAAGGGCAG CGCCAGCAGC GCACGCCTCC CCCTCCGGCG | |
| | GCCGCCACGT AGCGGGGAGC GCGCTGCAGC TCGGGCCTGG | |
| | GGCCTCGCGG CGGCGGCGGC GGCTCTAACG TTATCCGCGC | |
| | CCGCCGGCCC GCGGCGCGGC TGGGGGAGGG GACCCGCGCG | |
| | CTTTGTCTGG AGGCCCCGCA GCTGTCATCG CTCTCCCACC | |
| | CTCGGCACGA CTGTGCCGTC CTCCCCCGTG CAGCGGCGCG | |
| | GCGCGGCGCG GCGCGGCGCG GCGGCGCCTC AGCCAGGCCC | |
| | CGCCAAGCCT CCTCCCCGCA GCAGCCCCGG GTCCCGGGGG | |
| | CCCACGGCGG GGCGCGGGGG GCCCGCAGCC TCCCCGCAGG | |
| | GAAGCCGGGC GCACGGAGCA TCCCCGGCCC CGCCGCGCCG | |
| | CGCCGCGCTG CGCCACGCGC GACCCAGAAA AGCAAGGCAC | |
| | CGACTTCACC GCTCAGATGC TCCGGGCGCG GATCCTGCAG | |
| | AGTCTCCCGG CTGCGCGCCT CAGCTCAGCC ACATCCGAGG | |
| | GGAGGGGGCG CGGGCGCGGG CGCGGGCGCG GGCGGCTCGG | |
| | CCGGGGGGGA GGGGAGGGGA GGCACCGCCC ACGCCCAGGC | |
| | CGCGCGGGGC CCGCCGGGAA CGCGTCCTGC GGGGGGCGGC | |
| | GTGCGCGCCA CTCACCGTCC GCGGCGTGGC GCCTTGGGGG | |
| | TCTCCTGAGG GGAGGCGGGC GGGACCGCCG AGGCCGCTCT | |
| | GCGTCAAAGC CCAGCGCGCC AAAGCCGGGC TCCGCCCACC | |
| | TCCTCGCCCC GGGGCCTGCG GGGCTGGGGA GCTTCTGAGC | |
| | ACCGCCTCTC ACGGGGCGCG GGGGTCCCAG GGCCTCGGGT | |
| | GCCGCCGCGG CAGCCGCGCT CGGACTGCAC CCCCCCTGGG | |
| | GGACGGGCAC AAACAAACAA AGGCCGGCGC GCCTCGAGAG | |
| | GCCCGCAGCG AGGATCGGCG AAGGCGTCGT CGCCGCAGGG | |
| | AGTGCGGCCG GGGTGACGAG GGCCTCACGC CTGAGCGGCG | |
| | ACGCAAGCCC CGCCCGGCAG CGTTAATTTC AAACTGCGCG | |
| | ACCGTTTCTC CGGCTCCTTG CGCGGAGGTG TGGGGGGGGA | |
| | GGGCCGGACC TCACACCGGA CGTAGTAACA CGTTGGGCGC | |
| | GCGAGCCTTC CGCAATTCAC CAAACGCGGT TGCGCAAGTG | |
| | ACGTAACGCG GCCTCGCTAT CCCGGGAGGG GGAGGGCGCA | |
| | CGCACCCGGC GCAAAAGGCG TGTCGAGGCC CTTCCCGCCA | |
| | TTGGCGGCGG TTAGGACGTT TGCGTAACGG CCTGACGTAG | |
| | GCGGAGACGC GTTAGTGGGG GGAAGGTTCT AGAAAAGCGG | |
| | CGGTCCCGGC TCCAGCGGCA GTAGCAGCGG CGCCGGGTCC | |
| | TGTGTGCAGG TCTTTCCCCC GCGGCGCGGT TTCTCGAGCG | |
| | GCGGCTGCTC TTCCCCCCCC ACAGCGTCAG GACGAGTCCC | |
| | GTGCGTGTGC GTCCGCGGAG GTCTTTCTCA TCTCGCTCGG | |
| | CTGCGGGAAA TCGGGCTGAA GCGACTGAGT CCGCGATGGA | |
| | GGTAACGGGT TTGAAATCAA TGAGTTGTTG TTGGCGAGGG | |
| | CATGGCGAGG CCGCCGTGGA AGCCGGCCAC CGCCGCGGCG | |
| | GGCGGGCGGG CGGAGGGAGG GAGAGCGGCC GCCCGAGCGG | |
| | CGGTTGGCCC GGGGGCTCTG AGAGGGTCGT TGGCGGAGGC | |
| | CCGATTGTCC GAACGCCTTC CCCGCCCTCA GCCGCCCGGC | |
| | GCCATTTCCC TGAGCCGGGT GGGGGATGGG GCCCGGCCGC | |
| | GGCCAGCGGC TCGTCCTCCG CAGGGGCGCG GGAGCCCGGC | |
| | CGGCCTGGTT CCCCCGCGGG CCGTGCGCGG GGCCCCGGGG | |
| | CCCGGGGCGT GTGCGCGGCG GAGCGTTCGT TCGGGCGGCC | |
| | ACCGGGTCCC CCTGGAGCGG CTCTGCGCGG CTCCCCTCCC | |
| | CCTCCCTCCC TCCTCCCCCC CGGGAACCGG ATTTGGCGGC | |
| | CGCCATTTTC CCGTCTCCTT GCTCGCAGCG GTTTTTCGCG | |
| | TCCCGCACTC TGCGGCTCTG CCCGTTCGGC CCCTTCCCCC | |
| | CCGCGTGGGA GCCATTAGCT GGTTTCCTTT GTCCCCGGCC | |
| | CGTTTTGGTG GCGTTCCGAG GGGGATCTGG GGTGTTTTGA | |
| | GCTCTGATCT CGGAGTCCTG CGCACCTCGT TTTAGGCGCT | |
| | CCGCCCTCCT TCCCCCTCGA CGAAATGGCG CCATTGCGCT | |
| | CCCGAGCCAC ACGGTGTGCG CGGCCGGGTG GGGGTGGCGG | |
| | CGGCCTCCGC AAGGGTTCCC GGCGCAGGCC CCGGGCGAGG | |
| | AGTGCGATCC GGGCCTGAGC CTGCTGCTGA AGGCAGCCTT | |
| | GCAGCAAAGA AACCATTTTA CTTCCCCGAG CTGGCTCCTA | |
| | ATTC | |
| Plasmid 1 | AGATCTATTC GAGCTCGCCC GACATTGATT ATTGACTAGT | 4 |
| | TATTAATAGT AATCAATTAC GGGGTCATTA GTTCATAGCC | |
| | CATATATGGA GTTCCGCGTT ACATAACTTA CGGTAAATGG | |
| | CCCGCCTGGC TGACCGCCCA ACGACCCCCG CCCATTGACG | |
| | TCAATAATGA CGTATGTTCC CATAGTAACG CCAATAGGGA | |
| | CTTTCCATTG ACGTCAATGG GTGGAGTATT TACGGTAAAC | |
| | TGCCCACTTG GCAGTACATC AAGTGTATCA TATGCCAAGT | |
| | ACGCCCCCTA TTGACGTCAA TGACGGTAAA TGGCCCGCCT | |
| | GGCATTATGC CCAGTACATG ACCTTATGGG ACTTTCCTAC | |
| | TTGGCAGTAC ATCTACGTAT TAGTCATCGC TATTACCATG | |
| | GTGATGCGGT TTTGGCAGTA CATCAATGGG CGTGGATAGC | |
| | GGTTTGACTC ACGGGGATTT CCAAGTCTCC ACCCCATTGA | |
| | CGTCAATGGG AGTTTGTTTT GGCACCAAAA TCAACGGGAC | |
| | TTTCCAAAAT GTCGTAACAA CTCCGCCCCA TTGACGCAAA | |
| | TGGGCGGTAG GCGTGTACGG TGGGAGGTCT ATATAAGCAG | |
| | AGCTCGTTTA GTGAACCGTC AGATCGCCTG GAGACGCCAT | |
| | CCACGCTGTT TTGACCTCCA TAGAAGACAC CGGGACCGAT | |
| | CCAGCCTCCG CGGCCGGGAA CGGTGCATTG GAACGCGGAT | |
| | TCCCCGTGCC AAGAGTGACG TAAGTACCGC CTATAGAGTC | |
| | TATAGGCCCA CCCCCTTGGC TTCGTTAGAA CGCGGCTACA | |
| | ATTAATACAT AACCTTGTGT ATCATACACA TACGATTTAG | |
| | GTGACACTAT AGAATAACAT CCACTTTGCC TTTCTCTCCA | |
| | CAGGTGTCCA CTCCCAGGTC CAACTGCACC TCGGTTCTAT | |
| | CGATTGAATT CGCTAGCGCC ACCATGTCTA AGGGAGAGGA | |
| | GCTGTTCACC GGAGTGGTGC CAATCCTGGT GGAGCTGGAC | |
| | GGCGATGTGA ATGGCCACAA GTTTTCTGTG AGCGGAGAGG | |
| | GAGAGGGCGA CGCAACCTAC GGCAAGCTGA CACTGAAGTT | |
| | CATCTGCACC ACAGGCAAGC TGCCCGTGCC TTGGCCAACC | |
| | CTGGTGACCA CATTCACATA CGGCGTGCAG TGTTTTTCCA | |
| | GGTATCCTGA CCACATGAAG CAGCACGATT TCTTTAAGTC | |
| | TGCCATGCCA GAGGGCTACG TGCAGGAGCG GACCATCTTC | |
| | TTTAAGGACG ATGGCAATTA TAAGACCAGA GCCGAGGTGA | |
| | AGTTCGAGGG CGACACACTG GTGAACAGGA TCGAGCTGAA | |
| | GGGCATCGAC TTTAAGGAGG ATGGCAATAT CCTGGGCCAC | |
| | AAGCTGGAGT ACAACTATAA TAGCCACAAC GTGTACATCA | |
| | TGGCCGATAA GCAGAAGAAC GGCATCAAGG CTAACTTCAA | |
| | GATCCGCCAC AACATCGAGG ACGGCTCCGT GCAGCTGGCC | |
| | GATCACTACC AGCAGAACAC CCCAATCGGC GACGGACCCG | |
| | TGCTGCTGCC TGATAATCAC TATCTGTCCA CACAGTCTGC | |
| | CCTGAGCAAG GACCCTAATG AGAAGCGGGA TCACATGGTG | |
| | CTGCTGGAGT TTGTGACCGC AGCAGGAATC ACACACGGAA | |
| | TGGACGAGCT GTATAAGTAA AAGCTTGGCC GCCATGGCCC | |
| | AACTTGTTTA TTGCAGCTTA TAATGGTTAC AAATAAAGCA | |
| | ATAGCATCAC AAATTTCACA AATAAAGCAT TTTTTTCACT | |
| | GCATTCTAGT TGTGGTTTGT CCAAACTCAT CAATGTATCT | |
| | TATCATGTCT GGATCGGGAA TTAATTCGGC GCAGCACCAT | |
| | GGCCTGAAAT AACCTCTGAA AGAGGAACTT GGTTAGGTAC | |
| | CTTCTGAGGC GGAAAGAACC ATCTGTGGAA TGTGTGTCAG | |
| | TTAGGGTGTG GAAAGTCCCC AGGCTCCCCA GCAGGCAGAA | |
| | GTATGCAAAG CATGCATCTC AATTAGTCAG CAACCAGGTG | |
| | TGGAAAGTCC CCAGGCTCCC CAGCAGGCAG AAGTATGCAA | |
| | AGCATGCATC TCAATTAGTC AGCAACCATA GTCCCGCCCC | |
| | TAACTCCGCC CATCCCGCCC CTAACTCCGC CCAGTTCCGC | |
| | CCATTCTCCG CCCCATGGCT GACTAATTTT TTTTATTTAT | |
| | GCAGAGGCCG AGGCCGCCTC GGCCTCTGAG CTATTCCAGA | |
| | AGTAGTGAGG AGGCTTTTTT GGAGGCCTAG GCTTTTGCAA | |
| | AAAGCTGTTA ACAGCTTCTA GAGGAAAGAA CATGTGAGCA | |
| | AAAGGCCAGC AAAAGGCCAG GAACCGTAAA AAGGCCGCGT | |
| | TGCTGGCGTT TTTCCATAGG CTCCGCCCCC CTGACGAGCA | |
| | TCACAAAAAT CGACGCTCAA GTCAGAGGTG GCGAAACCCG | |
| | ACAGGACTAT AAAGATACCA GGCGTTTCCC CCTGGAAGCT | |
| | CCCTCGTGCG CTCTCCTGTT CCGACCCTGC CGCTTACCGG | |
| | ATACCTGTCC GCCTTTCTCC CTTCGGGAAG CGTGGCGCTT | |
| | TCTCATAGCT CACGCTGTAG GTATCTCAGT TCGGTGTAGG | |
| | TCGTTCGCTC CAAGCTGGGC TGTGTGCACG AACCCCCCGT | |
| | TCAGCCCGAC CGCTGCGCCT TATCCGGTAA CTATCGTCTT | |
| | GAGTCCAACC CGGTAAGACA CGACTTATCG CCACTGGCAG | |
| | CAGCCACTGG TAACAGGATT AGCAGAGCGA GGTATGTAGG | |
| | CGGTGCTACA GAGTTCTTGA AGTGGTGGCC TAACTACGGC | |
| | TACACTAGAA GAACAGTATT TGGTATCTGC GCTCTGCTGA | |
| | AGCCAGTTAC CTTCGGAAAA AGAGTTGGTA GCTCTTGATC | |
| | CGGCAAACAA ACCACCGCTG GTAGCGGTGG TTTTTTTGTT | |
| | TGCAAGCAGC AGATTACGCG CAGAAAAAAA GGATCTCAAG | |
| | AAGATCCTTT GATCTTTTCT ACGGGGTCTG ACGCTCAGTG | |
| | GAACGAAAAC TCACGTTAAG GGATTTTGGT CATGAGATTA | |
| | TCAAAAAGGA TCTTCACCTA GATCCTTTTA AATTAAAAAT | |
| | GAAGTTTTAA ATCAATCTAA AGTATATATG AGTAAACTTG | |
| | GTCTGACAGT TACCAATGCT TAATCAGTGA GGCACCTATC | |
| | TCAGCGATCT GTCTATTTCG TTCATCCATA GTTGCCTGAC | |
| | TCCCCGTCGT GTAGATAACT ACGATACGGG AGGGCTTACC | |
| | ATCTGGCCCC AGTGCTGCAA TGATACCGCG AGACCCACGC | |
| | TCACCGGCTC CAGATTTATC AGCAATAAAC CAGCCAGCCG | |
| | GAAGGGCCGA GCGCAGAAGT GGTCCTGCAA CTTTATCCGC | |
| | CTCCATCCAG TCTATTAATT GTTGCCGGGA AGCTAGAGTA | |
| | AGTAGTTCGC CAGTTAATAG TTTGCGCAAC GTTGTTGCCA | |
| | TTGCTACAGG CATCGTGGTG TCACGCTCGT CGTTTGGTAT | |
| | GGCTTCATTC AGCTCCGGTT CCCAACGATC AAGGCGAGTT | |
| | ACATGATCCC CCATGTTGTG CAAAAAAGCG GTTAGCTCCT | |
| | TCGGTCCTCC GATCGTTGTC AGAAGTAAGT TGGCCGCAGT | |
| | GTTATCACTC ATGGTTATGG CAGCACTGCA TAATTCTCTT | |
| | ACTGTCATGC CATCCGTAAG ATGCTTTTCT GTGACTGGTG | |
| | AGTACTCAAC CAAGTCATTC TGAGAATAGT GTATGCGGCG | |
| | ACCGAGTTGC TCTTGCCCGG CGTCAATACG GGATAATACC | |
| | GCGCCACATA GCAGAACTTT AAAAGTGCTC ATCATTGGAA | |
| | AACGTTCTTC GGGGCGAAAA CTCTCAAGGA TCTTACCGCT | |
| | GTTGAGATCC AGTTCGATGT AACCCACTCG TGCACCCAAC | |
| | TGATCTTCAG CATCTTTTAC TTTCACCAGC GTTTCTGGGT | |
| | GAGCAAAAAC AGGAAGGCAA AATGCCGCAA AAAAGGGAAT | |
| | AAGGGCGACA CGGAAATGTT GAATACTCAT ACTCTTCCTT | |
| | TTTCAATATT ATTGAAGCAT TTATCAGGGT TATTGTCTCA | |
| | TGAGCGGATA CATATTTGAA TGTATTTAGA AAAATAAACA | |
| | AATAGGGGTT CCGCGCACAT TTCCCCGAAA AGTGCCACCT | |
| | GACGTCCCAA T | |
| Plasmid 2 | AGATCTATTC GAGCTCGCCC GACATTGATT ATTGACTAGT | 5 |
| | TATTAATAGT AATCAATTAC GGGGTCATTA GTTCATAGCC | |
| | CATATATGGA GTTCCGCGTT ACATAACTTA CGGTAAATGG | |
| | CCCGCCTGGC TGACCGCCCA ACGACCCCCG CCCATTGACG | |
| | TCAATAATGA CGTATGTTCC CATAGTAACG CCAATAGGGA | |
| | CTTTCCATTG ACGTCAATGG GTGGAGTATT TACGGTAAAC | |
| | TGCCCACTTG GCAGTACATC AAGTGTATCA TATGCCAAGT | |
| | ACGCCCCCTA TTGACGTCAA TGACGGTAAA TGGCCCGCCT | |
| | GGCATTATGC CCAGTACATG ACCTTATGGG ACTTTCCTAC | |
| | TTGGCAGTAC ATCTACGTAT TAGTCATCGC TATTACCATG | |
| | GTGATGCGGT TTTGGCAGTA CATCAATGGG CGTGGATAGC | |
| | GGTTTGACTC ACGGGGATTT CCAAGTCTCC ACCCCATTGA | |
| | CGTCAATGGG AGTTTGTTTT GGCACCAAAA TCAACGGGAC | |
| | TTTCCAAAAT GTCGTAACAA CTCCGCCCCA TTGACGCAAA | |
| | TGGGCGGTAG GCGTGTACGG TGGGAGGTCT ATATAAGCAG | |
| | AGCTCGTTTA GTGAACCGTC AGATCGCCTG GAGACGCCAT | |
| | CCACGCTGTT TTGACCTCCA TAGAAGACAC CGGGACCGAT | |
| | CCAGCCTCCG CGGCCGGGAA CGGTGCATTG GAACGCGGAT | |
| | TCCCCGTGCC AAGAGTGACG TAAGTACCGC CTATAGAGTC | |
| | TATAGGCCCA CCCCCTTGGC TTCGTTAGAA CGCGGCTACA | |
| | ATTAATACAT AACCTTGTGT ATCATACACA TACGATTTAG | |
| | GTGACACTAT AGAATAACAT CCACTTTGCC TTTCTCTCCA | |
| | CAGGTGTCCA CTCCCAGGTC CAACTGCACC TCGGTTCTAT | |
| | CGATTGAATT CGCTAGCGCC ACCATGTCTA AGGGAGAGGA | |
| | GCTGTTCACC GGAGTGGTGC CAATCCTGGT GGAGCTGGAC | |
| | GGCGATGTGA ATGGCCACAA GTTTTCTGTG AGCGGAGAGG | |
| | GAGAGGGCGA CGCAACCTAC GGCAAGCTGA CACTGAAGTT | |
| | CATCTGCACC ACAGGCAAGC TGCCCGTGCC TTGGCCAACC | |
| | CTGGTGACCA CATTCACATA CGGCGTGCAG TGTTTTTCCA | |
| | GGTATCCTGA CCACATGAAG CAGCACGATT TCTTTAAGTC | |
| | TGCCATGCCA GAGGGCTACG TGCAGGAGCG GACCATCTTC | |
| | TTTAAGGACG ATGGCAATTA TAAGACCAGA GCCGAGGTGA | |
| | AGTTCGAGGG CGACACACTG GTGAACAGGA TCGAGCTGAA | |
| | GGGCATCGAC TTTAAGGAGG ATGGCAATAT CCTGGGCCAC | |
| | AAGCTGGAGT ACAACTATAA TAGCCACAAC GTGTACATCA | |
| | TGGCCGATAA GCAGAAGAAC GGCATCAAGG CTAACTTCAA | |
| | GATCCGCCAC AACATCGAGG ACGGCTCCGT GCAGCTGGCC | |
| | GATCACTACC AGCAGAACAC CCCAATCGGC GACGGACCCG | |
| | TGCTGCTGCC TGATAATCAC TATCTGTCCA CACAGTCTGC | |
| | CCTGAGCAAG GACCCTAATG AGAAGCGGGA TCACATGGTG | |
| | CTGCTGGAGT TTGTGACCGC AGCAGGAATC ACACACGGAA | |
| | TGGACGAGCT GTATAAGTAA AAGCTTGGCC GCCATGGCCC | |
| | AACTTGTTTA TTGCAGCTTA TAATGGTTAC AAATAAAGCA | |
| | ATAGCATCAC AAATTTCACA AATAAAGCAT TTTTTTCACT | |
| | GCATTCTAGT TGTGGTTTGT CCAAACTCAT CAATGTATCT | |
| | TATCATGTCT GGATCGGGAA TTAATTCGGC GCAGCACCAT | |
| | GGCCTGAAAT AACCTCTGAA AGAGGAACTT GGTTAGGTAC | |
| | CTTCTGAGGC GGAAAGAACC ATCTGTGGAA TGTGTGTCAG | |
| | TTAGGGTGTG GAAAGTCCCC AGGCTCCCCA GCAGGCAGAA | |
| | GTATGCAAAG CATGCATCTC AATTAGTCAG CAACCAGGTG | |
| | TGGAAAGTCC CCAGGCTCCC CAGCAGGCAG AAGTATGCAA | |
| | AGCATGCATC TCAATTAGTC AGCAACCATA GTCCCGCCCC | |
| | TAACTCCGCC CATCCCGCCC CTAACTCCGC CCAGTTCCGC | |
| | CCATTCTCCG CCCCATGGCT GACTAATTTT TTTTATTTAT | |
| | GCAGAGGCCG AGGCCGCCTC GGCCTCTGAG CTATTCCAGA | |
| | AGTAGTGAGG AGGCTTTTTT GGAGGCCTAG GCTTTTGCAA | |
| | AAAGCTGTTA ACAGCTTCTA GAGGAAAGAA CATGTGAGCA | |
| | AAAGGCCAGC AAAAGGCCAG GAACCGTAAA AAGGCCGCGT | |
| | TGCTGGCGTT TTTCCATAGG CTCCGCCCCC CTGACGAGCA | |
| | TCACAAAAAT CGACGCTCAA GTCAGAGGTG GCGAAACCCG | |
| | ACAGGACTAT AAAGATACCA GGCGTTTCCC CCTGGAAGCT | |
| | CCCTCGTGCG CTCTCCTGTT CCGACCCTGC CGCTTACCGG | |
| | ATACCTGTCC GCCTTTCTCC CTTCGGGAAG CGTGGCGCTT | |
| | TCTCATAGCT CACGCTGTAG GTATCTCAGT TCGGTGTAGG | |
| | TCGTTCGCTC CAAGCTGGGC TGTGTGCACG AACCCCCCGT | |
| | TCAGCCCGAC CGCTGCGCCT TATCCGGTAA CTATCGTCTT | |
| | GAGTCCAACC CGGTAAGACA CGACTTATCG CCACTGGCAG | |
| | CAGCCACTGG TAACAGGATT AGCAGAGCGA GGTATGTAGG | |
| | CGGTGCTACA GAGTTCTTGA AGTGGTGGCC TAACTACGGC | |
| | TACACTAGAA GAACAGTATT TGGTATCTGC GCTCTGCTGA | |
| | AGCCAGTTAC CTTCGGAAAA AGAGTTGGTA GCTCTTGATC | |
| | CGGCAAACAA ACCACCGCTG GTAGCGGTGG TTTTTTTGTT | |
| | TGCAAGCAGC AGATTACGCG CAGAAAAAAA GGATCTCAAG | |
| | AAGATCCTTT GATCTTTTCT ACGGGGTCTG ACGCTCAGTG | |
| | GAACGAAAAC TCACGTTAAG GGATTTTGGT CATGAGATTA | |
| | TCAAAAAGGA TCTTCACCTA GATCCTTTTA AATTAAAAAT | |
| | GAAGTTTTAA ATCAATCTAA AGTATATATG AGTAAACTTG | |
| | GTCTGACAGT TACCAATGCT TAATCAGTGA GGCACCTATC | |
| | TCAGCGATCT GTCTATTTCG TTCATCCATA GTTGCCTGAC | |
| | TCCCCGTCGT GTAGATAACT ACGATACGGG AGGGCTTACC | |
| | ATCTGGCCCC AGTGCTGCAA TGATACCGCG AGACCCACGC | |
| | TCACCGGCTC CAGATTTATC AGCAATAAAC CAGCCAGCCG | |
| | GAAGGGCCGA GCGCAGAAGT GGTCCTGCAA CTTTATCCGC | |
| | CTCCATCCAG TCTATTAATT GTTGCCGGGA AGCTAGAGTA | |
| | AGTAGTTCGC CAGTTAATAG TTTGCGCAAC GTTGTTGCCA | |
| | TTGCTACAGG CATCGTGGTG TCACGCTCGT CGTTTGGTAT | |
| | GGCTTCATTC AGCTCCGGTT CCCAACGATC AAGGCGAGTT | |
| | ACATGATCCC CCATGTTGTG CAAAAAAGCG GTTAGCTCCT | |
| | TCGGTCCTCC GATCGTTGTC AGAAGTAAGT TGGCCGCAGT | |
| | GTTATCACTC ATGGTTATGG CAGCACTGCA TAATTCTCTT | |
| | ACTGTCATGC CATCCGTAAG ATGCTTTTCT GTGACTGGTG | |
| | AGTACTCAAC CAAGTCATTC TGAGAATAGT GTATGCGGCG | |
| | ACCGAGTTGC TCTTGCCCGG CGTCAATACG GGATAATACC | |
| | GCGCCACATA GCAGAACTTT AAAAGTGCTC ATCATTGGAA | |
| | AACGTTCTTC GGGGCGAAAA CTCTCAAGGA TCTTACCGCT | |
| | GTTGAGATCC AGTTCGATGT AACCCACTCG TGCACCCAAC | |
| | TGATCTTCAG CATCTTTTAC TTTCACCAGC GTTTCTGGGT | |
| | GAGCAAAAAC AGGAAGGCAA AATGCCGCAA AAAAGGGAAT | |
| | AAGGGCGACA CGGAAATGTT GAATACTCAT ACTCTTCCTT | |
| | TTTCAATATT ATTGAAGCAT TTATCAGGGT TATTGTCTCA | |
| | TGAGCGGATA CATATTTGAA TGTATTTAGA AAAATAAACA | |
| | AATAGGGGTT CCGCGCACAT TTCCCCGAAA AGTGCCACCT | |
| | GACGTCCCAA TAGATCTGGC TTTCCACCTA GGGAAGGAAT | |
| | ACTCCTGCCT GCCTGCCTGC CTGGATCCTG CCAGAGGACG | |
| | GATGAAGCTG GAAGGAACCG CCGTGCTCCG CGCCCGCTCC | |
| | CCTTCCCCGC TCGTTGCCTG GTCACAGGCC GCGGCTGGGA | |
| | GCCCGCCCCC CCCCACTCGG GAGCGCGGGC CTCGTGGTCA | |
| | GCGCCTCCGC GGGGAGAAAC AAAGGAGGCG GCGGCGGCTC | |
| | AAGGGCAGCG CCAGCAGCGC ACGCCTCCCC CTCCGGCGGC | |
| | CGCCACGTAG CGGGGAGCGC GCTGCAGCTC GGGCCTGGGG | |
| | CCTCGCGGCG GCGGCGGCGG CTCTAACGTT ATCCGCGCCC | |
| | GCCGGCCCGC GGCGCGGCTG GGGGAGGGGA CCCGCGCGCT | |
| | TTGTCTGGAG GCCCCGCAGC TGTCATCGCT CTCCCACCCT | |
| | CGGCACGACT GTGCCGTCCT CCCCCGTGCA GCGGCGCGGC | |
| | GCGGCGCGGC GCGGCGCGGC GGCGCCTCAG CCAGGCCCCG | |
| | CCAAGCCTCC TCCCCGCAGC AGCCCCGGGT CCCGGGGGCC | |
| | CACGGCGGGG CGCGGGGGGC CCGCAGCCTC CCCGCAGGGA | |
| | AGCCGGGCGC ACGGAGCATC CCCGGCCCCG CCGCGCCGCG | |
| | CCGCGCTGCG CCACGCGCGA CCCAGAAAAG CAAGGCACCG | |
| | ACTTCACCGC TCAGATGCTC CGGGCGCGGA TCCTGCAGAG | |
| | TCTCCCGGCT GCGCGCCTCA GCTCAGCCAC ATCCGAGGGG | |
| | AGGGGGCGCG GGCGCGGGCG CGGGCGCGGG CGGCTCGGCC | |
| | GGGGGGGAGG GGAGGGGAGG CACCGCCCAC GCCCAGGCCG | |
| | CGCGGGGCCC GCCGGGAACG CGTCCTGCGG GGGGCGGCGT | |
| | GCGCGCCACT CACCGTCCGC GGCGTGGCGC CTTGGGGGTC | |
| | TCCTGAGGGG AGGCGGGCGG GACCGCCGAG GCCGCTCTGC | |
| | GTCAAAGCCC AGCGCGCCAA AGCCGGGCTC CGCCCACCTC | |
| | CTCGCCCCGG GGCCTGCGGG GCTGGGGAGC TTCTGAGCAC | |
| | CGCCTCTCAC GGGGCGCGGG GGTCCCAGGG CCTCGGGTGC | |
| | CGCCGCGGCA GCCGCGCTCG GACTGCACCC CCCCTGGGGG | |
| | ACGGGCACAA ACAAACAAAG GCCGGCGCGC CTCGAGAGGC | |
| | CCGCAGCGAG GATCGGCGAA GGCGTCGTCG CCGCAGGGAG | |
| | TGCGGCCGGG GTGACGAGGG CCTCACGCCT GAGCGGCGAC | |
| | GCAAGCCCCG CCCGGCAGCG TTAATTTCAA ACTGCGCGAC | |
| | CGTTTCTCCG GCTCCTTGCG CGGAGGTGTG GGGGGGGAGG | |
| | GCCGGACCTC ACACCGGACG TAGTAACACG TTGGGCGCGC | |
| | GAGCCTTCCG CAATTCACCA AACGCGGTTG CGCAAGTGAC | |
| | GTAACGCGGC CTCGCTATCC CGGGAGGGGG AGGGCGCACG | |
| | CACCCGGCGC AAAAGGCGTG TCGAGGCCCT TCCCGCCATT | |
| | GGCGGCGGTT AGGACGTTTG CGTAACGGCC TGACGTAGGC | |
| | GGAGACGCGT TAGTGGGGGG AAGGTTCTAG AAAAGCGGCG | |
| | GTCCCGGCTC CAGCGGCAGT AGCAGCGGCG CCGGGTCCTG | |
| | TGTGCAGGTC TTTCCCCCGC GGCGCGGTTT CTCGAGCGGC | |
| | GGCTGCTCTT CCCCCCCCAC AGCGTCAGGA CGAGTCCCGT | |
| | GCGTGTGCGT CCGCGGAGGT CTTTCTCATC TCGCTCGGCT | |
| | GCGGGAAATC GGGCTGAAGC GACTGAGTCC GCGATGGAGG | |
| | TAACGGGTTT GAAATCAATG AGTTGTTGTT GGCGAGGGCA | |
| | TGGCGAGGCC GCCGTGGAAG CCGGCCACCG CCGCGGCGGG | |
| | CGGGCGGGCG GAGGGAGGGA GAGCGGCCGC CCGAGCGGCG | |
| | GTTGGCCCGG GGGCTCTGAG AGGGTCGTTG GCGGAGGCCC | |
| | GATTGTCCGA ACGCCTTCCC CGCCCTCAGC CGCCCGGCGC | |
| | CATTTCCCTG AGCCGGGTGG GGGATGGGGC CCGGCCGCGG | |
| | CCAGCGGCTC GTCCTCCGCA GGGGCGCGGG AGCCCGGCCG | |
| | GCCTGGTTCC CCCGCGGGCC GTGCGCGGGG CCCCGGGGCC | |
| | CGGGGCGTGT GCGCGGCGGA GCGTTCGTTC GGGCGGCCAC | |
| | CGGGTCCCCC TGGAGCGGCT CTGCGCGGCT CCCCTCCCCC | |
| | TCCCTCCCTC CTCCCCCCCG GGAACCGGAT TTGGCGGCCG | |
| | CCATTTTCCC GTCTCCTTGC TCGCAGCGGT TTTTCGCGTC | |
| | CCGCACTCTG CGGCTCTGCC CGTTCGGCCC CTTCCCCCCC | |
| | GCGTGGGAGC CATTAGCTGG TTTCCTTTGT CCCCGGCCCG | |
| | TTTTGGTGGC GTTCCGAGGG GGATCTGGGG TGTTTTGAGC | |
| | TCTGATCTCG GAGTCCTGCG CACCTCGTTT TAGGCGCTCC | |
| | GCCCTCCTTC CCCCTCGACG AAATGGCGCC ATTGCGCTCC | |
| | CGAGCCACAC GGTGTGCGCG GCCGGGTGGG GGTGGCGGCG | |
| | GCCTCCGCAA GGGTTCCCGG CGCAGGCCCC GGGCGAGGAG | |
| | TGCGATCCGG GCCTGAGCCT GCTGCTGAAG GCAGCCTTGC | |
| | AGCAAAGAAA CCATTTTACT TCCCCGAGCT GGCTCCTAAT | |
| | TC | |

While preferred embodiments of the present disclosure have been shown and described herein, it will be obvious to those skilled in the art that such embodiments are provided by way of example only. Numerous variations, changes, and substitutions will now occur to those skilled in the art without departing from the disclosure. It should be understood that various alternatives to the embodiments of the disclosure described herein may be employed in practicing the disclosure. It is intended that the following claims define the scope of the disclosure and that methods and structures within the scope of these claims and their equivalents be covered thereby.

1.-34. (canceled)

35. An isolated polynucleotide comprising:
an extended methylation-free CpG island encompassing dual divergently transcribed promoters;
a target gene of interest adjacent to the extended methylation-free CpG island;
a polyadenylation signal located at the 3′ terminus of the target gene of interest; and wherein the GC content of the extended methylation-free CpG island over a 200 bp range is from about 62% to about 88%.

36. The isolated polynucleotide of claim 35, wherein the GC content of the extended methylation-free CpG island over a 200 bp range is from about 65% to about 85%.

37. The isolated polynucleotide of claim 35, wherein the GC content of the extended methylation-free CpG island over a 200 bp range is from about 70% to about 80%.

38. The isolated polynucleotide of claim 35, wherein the GC content of the extended methylation-free CpG island is higher than 62%.

39. The isolated polynucleotide of claim 38, wherein the GC content of the extended methylation-free CpG island is higher than 65%.

40. The isolated polynucleotide of claim 38, wherein the GC content of the extended methylation-free CpG island is higher than 70%.

41. The isolated polynucleotide of claim 35, wherein at least one of the dual divergently transcribed promoters comprises:
a) a constitutive promoter;
b) an inducible promoter;
c) a eukaryotic promoter;
d) a prokaryotic promoter; or
e) a viral promoter.

42. The isolated polynucleotide of claim 35, wherein the extended methylation-free CpG island comprises SEQ ID NO:1 or a nucleotide sequence having at least 85% sequence identity to SEQ ID NO: 1.

43. The isolated polynucleotide of claim 35, further comprising a selectable marker.

44. The isolated polynucleotide of claim 43, wherein the selectable marker is located adjacent to the extended methylation-free CpG island but at the opposing terminus from the target gene of interest.

45. The isolated polynucleotide of claim 43, wherein the selectable marker is located between the extended methylation-free CpG island and the target gene of interest.

46. The isolated polynucleotide of claim 43, wherein the selectable marker is located proximal to the polyadenylation signal.

47. The isolated polynucleotide of claim 46, wherein the selectable marker is more than 2000 bp from the proximal end of the polyadenylation signal.

48. The isolated polynucleotide of claim 43, wherein the selectable marker is an antibiotic resistance gene or a fluorescent protein.

49. The isolated polynucleotide of claim 43, wherein the selectable marker is selected from the group consisting of adenine deaminase (ada), blasticidin S deaminases (Bsr, BSD), bleomycin-binding protein (Ble), Neomycin phosphotransferase(neo), histidinol dehydrogenase (hisD), glutamine synthetase (GS) (also known as glutamine ammonia ligase or GLUL), dihydrofolate reductase (dhfr), cytosine deaminase (codA), puromycin N-acetyltransferase (Pac), and hygromycin B phosphotransferase (Hph).

50. The isolated polynucleotide of claim 43, wherein the selectable marker is selected from the group consisting of a green fluorescent protein, a blue fluorescent protein, a cyan fluorescent protein, a yellow fluorescent protein, an orange fluorescent protein, a red fluorescent protein, a far-Red fluorescent protein, a near-IR protein, a Fong Stokes Shift protein, a photoactivatable protein, a photoconvertible protein, a photoswitchable protein.

51. The isolated polynucleotide of claim 35, wherein the extended methylation-free CpG island comprises at least 90% sequence identity to at least 500 contiguous bases of SEQ ID NO:1.

52. A method of enhancing the expression of a target protein, comprising:
a) contacting a host cell with the isolated polynucleotide of claim 35, wherein the target gene of interest encodes the target protein; and
b) culturing the host cell at a sufficient condition wherein the host cell expresses the target protein, thereby inducing an enhanced expression of the target protein.

53. The method of claim 52, wherein the host cell is a eukaryotic host cell.

54. An isolated vector comprising SEQ ID NOs: 2, 3, or 5, or a sequence comprising 90% identity to any of SEQ ID NOs: 2, 3, or 5.


SEQUENCE LISTING (5 sequences)

SEQ ID NO: 1 - DNA, 2585 bp, Artificial Sequence
  Description of Artificial Sequence Synthetic polynucleotide
gaattaggagccagctcggggaagtaaaatggtttctttgctgcaaggctgccttcagcagcaggctcaggcccggatcgcactcctcgcccggggcctgcgccgggaacccttgcggaggccgccgccacccccacccggccgcgcacaccgtgtggctcgggagcgcaatggcgccatttcgtcgagggggaaggagggcggagcgcctaaaacgaggtgcgcaggactccgagatcagagctcaaaacaccccagatccccctcggaacgccaccaaaacgggccggggacaaaggaaaccagctaatggctcccacgcgggggggaaggggccgaacgggcagagccgcagagtgcgggacgcgaaaaaccgctgcgagcaaggagacgggaaaatggcggccgccaaatccggttcccgggggggaggagggagggagggggaggggagccgcgcagagccgctccagggggacccggtggccgcccgaacgaacgctccgccgcgcacacgccccgggccccggggccccgcgcacggcccgcgggggaaccaggccggccgggctcccgcgcccctgcggaggacgagccgctggccgcggccgggccccatcccccacccggctcagggaaatggcgccgggcggctgagggcggggaaggcgttcggacaatcgggcctccgccaacgaccctctcagagcccccgggccaaccgccgctcgggcggccgctctccctccctccgcccgcccgcccgccgcggcggtggccggcttccacggcggcctcgccatgccctcgccaacaacaactcattgatttcaaacccgttacctccatcgcggactcagtcgcttcagcccgatttcccgcagccgagcgagatgagaaagacctccgcggacgcacacgcacgggactcgtcctgacgctgtggggggggaagagcagccgccgctcgagaaaccgcgccgcgggggaaagacctgcacacaggacccggcgccgctgctactgccgctggagccgggaccgccgcttttctagaaccttccccccactaacgcgtctccgcctacgtcaggccgttacgcaaacgtcctaaccgccgccaatggcgggaagggcctcgacacgccttttgcgccgggtgcgtgcgccctccccctcccgggatagcgaggccgcgttacgtcacttgcgcaaccgcgtttggtgaattgcggaaggctcgcgcgcccaacgtgttactacgtccggtgtgaggtccggccctccccccccacacctccgcgcaaggagccggagaaacggtcgcgcagtttgaaattaacgctgccgggcggggcttgcgtcgccgctcaggcgtgaggccctcgtcaccccggccgcactccctgcggcgacgacgccttcgccgatcctcgctgcgggcctctcgaggcgcgccggcctttgtttgtttgtgcccgtcccccagggggggtgcagtccgagcgcggctgccgcggcggcacccgaggccctgggacccccgcgccccgtgagaggcggtgctcagaagctccccagccccgcaggccccggggcgaggaggtgggcggagcccggctttggcgcgctgggctttgacgcagagcggcctcggcggtcccgcccgcctcccctcaggagacccccaaggcgccacgccgcggacggtgagtggcgcgcacgccgccccccgcaggacgcgttcccggcgggccccgcgcggcctgggcgtgggcggtgcctcccctcccctcccccccggccgagccgcccgcgcccgcgcccgcgcccgcgccccctcccctcggatgtggctgagctgaggcgcgcagccgggagactctgcaggatccgcgcccggagcatctgagcggtgaagtcggtgccttgcttttctgggtcgcgcgtggcgcagcgcggcgcggcgcggcggggccggggatgctccgtgcgcccggcttccctgcggggaggctgcgggccccccgcgccccgccgtgggcccccgggacccggggctgctgcggggaggaggcttggcggggcctggctgaggcgccgccgcgccgcgccgcgccgcgccgcgccgctgcacgggggaggacggcacagtcgtgccgagggtgggagagcgatgacagctgcggggcctccagacaaagcgcgcgggtcccctcccccagccgcgccgcgggccggcgggcgcggataacgttagagccgccgccgccgccgcgaggccccaggcccgagctgcagcgcgctccccgctacgtggcggccgccggagggggaggcgtgcgctgctggcgctgcccttgagccgccgccgcctcctttgtttctccccgcggaggcgctgaccacgaggcccgcgctcccgagtgggggggggcgggctcccagccgcggcctgtgaccaggcaacgagcggggaaggggagcgggcgcggagcacggcggttccttccagcttcatccgtcctctggcaggatccaggcaggcaggcaggcaggagtattccttccctaggtggaaagcc

SEQ ID NO: 2 - DNA, 8004 bp, Artificial Sequence
  Description of Artificial Sequence Synthetic polynucleotide
agatctattcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttagaacgcggctacaattaatacataaccttgtgtatcatacacatacgatttaggtgacactatagaataacatccactttgcctttctctccacaggtgtccactcccaggtccaactgcacctcggttctatcgattgaattcgctagcgccaccatgtctaagggagaggagctgttcaccggagtggtgccaatcctggtggagctggacggcgatgtgaatggccacaagttttctgtgagcggagagggagagggcgacgcaacctacggcaagctgacactgaagttcatctgcaccacaggcaagctgcccgtgccttggccaaccctggtgaccacattcacatacggcgtgcagtgtttttccaggtatcctgaccacatgaagcagcacgatttctttaagtctgccatgccagagggctacgtgcaggagcggaccatcttctttaaggacgatggcaattataagaccagagccgaggtgaagttcgagggcgacacactggtgaacaggatcgagctgaagggcatcgactttaaggaggatggcaatatcctgggccacaagctggagtacaactataatagccacaacgtgtacatcatggccgataagcagaagaacggcatcaaggctaacttcaagatccgccacaacatcgaggacggctccgtgcagctggccgatcactaccagcagaacaccccaatcggcgacggacccgtgctgctgcctgataatcactatctgtccacacagtctgccctgagcaaggaccctaatgagaagcgggatcacatggtgctgctggagtttgtgaccgcagcaggaatcacacacggaatggacgagctgtataagtaaaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgggaattaattcggcgcagcaccatggcctgaaataacctctgaaagaggaacttggttaggtaccttctgaggcggaaagaaccatctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgttaacagcttctagaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcccaatagatccgaacaaacgacccaacacccgtgcgttttattctgtctttttattgccgtcatagcgcgggttccttccggtattgtctccttccgtgtttcagttagcctcccccatctcccgatccggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcggcacgctgttgacgctgttaagcgggtcgctgcagggtcgctcggtgttcgaggccacacgcgtcaccttaatatgcgaagtggacctcggaccgcgccgccccgactgcatctgcgtgttcgaattcgccaatgacaagacgctgggcggggtttgtgtcatagatctgaattaggagccagctcggggaagtaaaatggtttctttgctgcaaggctgccttcagcagcaggctcaggcccggatcgcactcctcgcccggggcctgcgccgggaacccttgcggaggccgccgccacccccacccggccgcgcacaccgtgtggctcgggagcgcaatggcgccatttcgtcgagggggaaggagggcggagcgcctaaaacgaggtgcgcaggactccgagatcagagctcaaaacaccccagatccccctcggaacgccaccaaaacgggccggggacaaaggaaaccagctaatggctcccacgcgggggggaaggggccgaacgggcagagccgcagagtgcgggacgcgaaaaaccgctgcgagcaaggagacgggaaaatggcggccgccaaatccggttcccgggggggaggagggagggagggggaggggagccgcgcagagccgctccagggggacccggtggccgcccgaacgaacgctccgccgcgcacacgccccgggccccggggccccgcgcacggcccgcgggggaaccaggccggccgggctcccgcgcccctgcggaggacgagccgctggccgcggccgggccccatcccccacccggctcagggaaatggcgccgggcggctgagggcggggaaggcgttcggacaatcgggcctccgccaacgaccctctcagagcccccgggccaaccgccgctcgggcggccgctctccctccctccgcccgcccgcccgccgcggcggtggccggcttccacggcggcctcgccatgccctcgccaacaacaactcattgatttcaaacccgttacctccatcgcggactcagtcgcttcagcccgatttcccgcagccgagcgagatgagaaagacctccgcggacgcacacgcacgggactcgtcctgacgctgtggggggggaagagcagccgccgctcgagaaaccgcgccgcgggggaaagacctgcacacaggacccggcgccgctgctactgccgctggagccgggaccgccgcttttctagaaccttccccccactaacgcgtctccgcctacgtcaggccgttacgcaaacgtcctaaccgccgccaatggcgggaagggcctcgacacgccttttgcgccgggtgcgtgcgccctccccctcccgggatagcgaggccgcgttacgtcacttgcgcaaccgcgtttggtgaattgcggaaggctcgcgcgcccaacgtgttactacgtccggtgtgaggtccggccctccccccccacacctccgcgcaaggagccggagaaacggtcgcgcagtttgaaattaacgctgccgggcggggcttgcgtcgccgctcaggcgtgaggccctcgtcaccccggccgcactccctgcggcgacgacgccttcgccgatcctcgctgcgggcctctcgaggcgcgccggcctttgtttgtttgtgcccgtcccccagggggggtgcagtccgagcgcggctgccgcggcggcacccgaggccctgggacccccgcgccccgtgagaggcggtgctcagaagctccccagccccgcaggccccggggcgaggaggtgggcggagcccggctttggcgcgctgggctttgacgcagagcggcctcggcggtcccgcccgcctcccctcaggagacccccaaggcgccacgccgcggacggtgagtggcgcgcacgccgccccccgcaggacgcgttcccggcgggccccgcgcggcctgggcgtgggcggtgcctcccctcccctcccccccggccgagccgcccgcgcccgcgcccgcgcccgcgccccctcccctcggatgtggctgagctgaggcgcgcagccgggagactctgcaggatccgcgcccggagcatctgagcggtgaagtcggtgccttgcttttctgggtcgcgcgtggcgcagcgcggcgcggcgcggcggggccggggatgctccgtgcgcccggcttccctgcggggaggctgcgggccccccgcgccccgccgtgggcccccgggacccggggctgctgcggggaggaggcttggcggggcctggctgaggcgccgccgcgccgcgccgcgccgcgccgcgccgctgcacgggggaggacggcacagtcgtgccgagggtgggagagcgatgacagctgcggggcctccagacaaagcgcgcgggtcccctcccccagccgcgccgcgggccggcgggcgcggataacgttagagccgccgccgccgccgcgaggccccaggcccgagctgcagcgcgctccccgctacgtggcggccgccggagggggaggcgtgcgctgctggcgctgcccttgagccgccgccgcctcctttgtttctccccgcggaggcgctgaccacgaggcccgcgctcccgagtgggggggggcgggctcccagccgcggcctgtgaccaggcaacgagcggggaaggggagcgggcgcggagcacggcggttccttccagcttcatccgtcctctggcaggatccaggcaggcaggcaggcaggagtattccttccctaggtggaaagcc

SEQ ID NO: 3 - DNA, 8004 bp, Artificial Sequence
  Description of Artificial Sequence Synthetic polynucleotide
agatctattcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttagaacgcggctacaattaatacataaccttgtgtatcatacacatacgatttaggtgacactatagaataacatccactttgcctttctctccacaggtgtccactcccaggtccaactgcacctcggttctatcgattgaattcgctagcgccaccatgtctaagggagaggagctgttcaccggagtggtgccaatcctggtggagctggacggcgatgtgaatggccacaagttttctgtgagcggagagggagagggcgacgcaacctacggcaagctgacactgaagttcatctgcaccacaggcaagctgcccgtgccttggccaaccctggtgaccacattcacatacggcgtgcagtgtttttccaggtatcctgaccacatgaagcagcacgatttctttaagtctgccatgccagagggctacgtgcaggagcggaccatcttctttaaggacgatggcaattataagaccagagccgaggtgaagttcgagggcgacacactggtgaacaggatcgagctgaagggcatcgactttaaggaggatggcaatatcctgggccacaagctggagtacaactataatagccacaacgtgtacatcatggccgataagcagaagaacggcatcaaggctaacttcaagatccgccacaacatcgaggacggctccgtgcagctggccgatcactaccagcagaacaccccaatcggcgacggacccgtgctgctgcctgataatcactatctgtccacacagtctgccctgagcaaggaccctaatgagaagcgggatcacatggtgctgctggagtttgtgaccgcagcaggaatcacacacggaatggacgagctgtataagtaaaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgggaattaattcggcgcagcaccatggcctgaaataacctctgaaagaggaacttggttaggtaccttctgaggcggaaagaaccatctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgttaacagcttctagaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcccaatagatccgaacaaacgacccaacacccgtgcgttttattctgtctttttattgccgtcatagcgcgggttccttccggtattgtctccttccgtgtttcagttagcctcccccatctcccgatccggacgagtgctggggcgtcggtttccactatcggcgagtacttctacacagccatcggtccagacggccgcgcttctgcgggcgatttgtgtacgcccgacagtcccggctccggatcggacgattgcgtcgcatcgaccctgcgcccaagctgcatcatcgaaattgccgtcaaccaagctctgatagagttggtcaagaccaatgcggagcatatacgcccggagccgcggcgatcctgcaagctccggatgcctccgctcgaagtagcgcgtctgctgctccatacaagccaaccacggcctccagaagaagatgttggcgacctcgtattgggaatccccgaacatcgcctcgctccagtcaatgaccgctgttatgcggccattgtccgtcaggacattgttggagccgaaatccgcgtgcacgaggtgccggacttcggggcagtcctcggcccaaagcatcagctcatcgagagcctgcgcgacggacgcactgacggtgtcgtccatcacagtttgccagtgatacacatggggatcagcaatcgcgcatatgaaatcacgccatgtagtgtattgaccgattccttgcggtccgaatgggccgaacccgctcgtctggctaagatcggccgcagcgatcgcatccatggcctccgcgaccggctgcagaacagcgggcagttcggtttcaggcaggtcttgcaacgtgacaccctgtgcacggcgggagatgcaataggtcaggctctcgctgaattccccaatgtcaagcacttccggaatcgggagcgcggccgatgcaaagtgccgataaacataacgatctttgtagaaaccatcggcgcagctatttacccgcaggacatatccacgccctcctacatcgaagctgaaagcacgagattcttcgccctccgagagctgcatcaggtcggagacgctgtcgaacttttcgatcagaaacttctcgacagacgtcgcggtgagttcaggctttttcatatctcattgccccccgggatctgcggcacgctgttgacgctgttaagcgggtcgctgcagggtcgctcggtgttcgaggccacacgcgtcaccttaatatgcgaagtggacctcggaccgcgccgccccgactgcatctgcgtgttcgaattcgccaatgacaagacgctgggcggggtttgtgtcatagatctggctttccacctagggaaggaatactcctgcctgcctgcctgcctggatcctgccagaggacggatgaagctggaaggaaccgccgtgctccgcgcccgctccccttccccgctcgttgcctggtcacaggccgcggctgggagcccgcccccccccactcgggagcgcgggcctcgtggtcagcgcctccgcggggagaaacaaaggaggcggcggcggctcaagggcagcgccagcagcgcacgcctccccctccggcggccgccacgtagcggggagcgcgctgcagctcgggcctggggcctcgcggcggcggcggcggctctaacgttatccgcgcccgccggcccgcggcgcggctgggggaggggacccgcgcgctttgtctggaggccccgcagctgtcatcgctctcccaccctcggcacgactgtgccgtcctcccccgtgcagcggcgcggcgcggcgcggcgcggcgcggcggcgcctcagccaggccccgccaagcctcctccccgcagcagccccgggtcccgggggcccacggcggggcgcggggggcccgcagcctccccgcagggaagccgggcgcacggagcatccccggccccgccgcgccgcgccgcgctgcgccacgcgcgacccagaaaagcaaggcaccgacttcaccgctcagatgctccgggcgcggatcctgcagagtctcccggctgcgcgcctcagctcagccacatccgaggggagggggcgcgggcgcgggcgcgggcgcgggcggctcggccgggggggaggggaggggaggcaccgcccacgcccaggccgcgcggggcccgccgggaacgcgtcctgcggggggcggcgtgcgcgccactcaccgtccgcggcgtggcgccttgggggtctcctgaggggaggcgggcgggaccgccgaggccgctctgcgtcaaagcccagcgcgccaaagccgggctccgcccacctcctcgccccggggcctgcggggctggggagcttctgagcaccgcctctcacggggcgcgggggtcccagggcctcgggtgccgccgcggcagccgcgctcggactgcaccccccctgggggacgggcacaaacaaacaaaggccggcgcgcctcgagaggcccgcagcgaggatcggcgaaggcgtcgtcgccgcagggagtgcggccggggtgacgagggcctcacgcctgagcggcgacgcaagccccgcccggcagcgttaatttcaaactgcgcgaccgtttctccggctccttgcgcggaggtgtggggggggagggccggacctcacaccggacgtagtaacacgttgggcgcgcgagccttccgcaattcaccaaacgcggttgcgcaagtgacgtaacgcggcctcgctatcccgggagggggagggcgcacgcacccggcgcaaaaggcgtgtcgaggcccttcccgccattggcggcggttaggacgtttgcgtaacggcctgacgtaggcggagacgcgttagtggggggaaggttctagaaaagcggcggtcccggctccagcggcagtagcagcggcgccgggtcctgtgtgcaggtctttcccccgcggcgcggtttctcgagcggcggctgctcttccccccccacagcgtcaggacgagtcccgtgcgtgtgcgtccgcggaggtctttctcatctcgctcggctgcgggaaatcgggctgaagcgactgagtccgcgatggaggtaacgggtttgaaatcaatgagttgttgttggcgagggcatggcgaggccgccgtggaagccggccaccgccgcggcgggcgggcgggcggagggagggagagcggccgcccgagcggcggttggcccgggggctctgagagggtcgttggcggaggcccgattgtccgaacgccttccccgccctcagccgcccggcgccatttccctgagccgggtgggggatggggcccggccgcggccagcggctcgtcctccgcaggggcgcgggagcccggccggcctggttcccccgcgggccgtgcgcggggccccggggcccggggcgtgtgcgcggcggagcgttcgttcgggcggccaccgggtccccctggagcggctctgcgcggctcccctccccctccctccctcctcccccccgggaaccggatttggcggccgccattttcccgtctccttgctcgcagcggtttttcgcgtcccgcactctgcggctctgcccgttcggccccttcccccccgcgtgggagccattagctggtttcctttgtccccggcccgttttggtggcgttccgagggggatctggggtgttttgagctctgatctcggagtcctgcgcacctcgttttaggcgctccgccctccttccccctcgacgaaatggcgccattgcgctcccgagccacacggtgtgcgcggccgggtgggggtggcggcggcctccgcaagggttcccggcgcaggccccgggcgaggagtgcgatccgggcctgagcctgctgctgaaggcagccttgcagcaaagaaaccattttacttccccgagctggctcctaattc

SEQ ID NO: 4 - DNA, 4091 bp, Artificial Sequence
  Description of Artificial Sequence Synthetic polynucleotide
agatctattcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttagaacgcggctacaattaatacataaccttgtgtatcatacacatacgatttaggtgacactatagaataacatccactttgcctttctctccacaggtgtccactcccaggtccaactgcacctcggttctatcgattgaattcgctagcgccaccatgtctaagggagaggagctgttcaccggagtggtgccaatcctggtggagctggacggcgatgtgaatggccacaagttttctgtgagcggagagggagagggcgacgcaacctacggcaagctgacactgaagttcatctgcaccacaggcaagctgcccgtgccttggccaaccctggtgaccacattcacatacggcgtgcagtgtttttccaggtatcctgaccacatgaagcagcacgatttctttaagtctgccatgccagagggctacgtgcaggagcggaccatcttctttaaggacgatggcaattataagaccagagccgaggtgaagttcgagggcgacacactggtgaacaggatcgagctgaagggcatcgactttaaggaggatggcaatatcctgggccacaagctggagtacaactataatagccacaacgtgtacatcatggccgataagcagaagaacggcatcaaggctaacttcaagatccgccacaacatcgaggacggctccgtgcagctggccgatcactaccagcagaacaccccaatcggcgacggacccgtgctgctgcctgataatcactatctgtccacacagtctgccctgagcaaggaccctaatgagaagcgggatcacatggtgctgctggagtttgtgaccgcagcaggaatcacacacggaatggacgagctgtataagtaaaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgggaattaattcggcgcagcaccatggcctgaaataacctctgaaagaggaacttggttaggtaccttctgaggcggaaagaaccatctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgttaacagcttctagaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcccaat

SEQ ID NO: 5 - DNA, 6682 bp, Artificial Sequence
  Description of Artificial Sequence Synthetic polynucleotide
agatctattcgagctcgcccgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcgttagaacgcggctacaattaatacataaccttgtgtatcatacacatacgatttaggtgacactatagaataacatccactttgcctttctctccacaggtgtccactcccaggtccaactgcacctcggttctatcgattgaattcgctagcgccaccatgtctaagggagaggagctgttcaccggagtggtgccaatcctggtggagctggacggcgatgtgaatggccacaagttttctgtgagcggagagggagagggcgacgcaacctacggcaagctgacactgaagttcatctgcaccacaggcaagctgcccgtgccttggccaaccctggtgaccacattcacatacggcgtgcagtgtttttccaggtatcctgaccacatgaagcagcacgatttctttaagtctgccatgccagagggctacgtgcaggagcggaccatcttctttaaggacgatggcaattataagaccagagccgaggtgaagttcgagggcgacacactggtgaacaggatcgagctgaagggcatcgactttaaggaggatggcaatatcctgggccacaagctggagtacaactataatagccacaacgtgtacatcatggccgataagcagaagaacggcatcaaggctaacttcaagatccgccacaacatcgaggacggctccgtgcagctggccgatcactaccagcagaacaccccaatcggcgacggacccgtgctgctgcctgataatcactatctgtccacacagtctgccctgagcaaggaccctaatgagaagcgggatcacatggtgctgctggagtttgtgaccgcagcaggaatcacacacggaatggacgagctgtataagtaaaagcttggccgccatggcccaacttgtttattgcagcttataatggttacaaataaagcaatagcatcacaaatttcacaaataaagcatttttttcactgcattctagttgtggtttgtccaaactcatcaatgtatcttatcatgtctggatcgggaattaattcggcgcagcaccatggcctgaaataacctctgaaagaggaacttggttaggtaccttctgaggcggaaagaaccatctgtggaatgtgtgtcagttagggtgtggaaagtccccaggctccccagcaggcagaagtatgcaaagcatgcatctcaattagtcagcaaccaggtgtggaaagtccccaggctccccagcaggcagaagtatgcaaagcatgcatctcaattagtcagcaaccatagtcccgcccctaactccgcccatcccgcccctaactccgcccagttccgcccattctccgccccatggctgactaattttttttatttatgcagaggccgaggccgcctcggcctctgagctattccagaagtagtgaggaggcttttttggaggcctaggcttttgcaaaaagctgttaacagcttctagaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagcagaactttaaaagtgctcatcattggaaaacgttcttcggggcgaaaactctcaaggatcttaccgctgttgagatccagttcgatgtaacccactcgtgcacccaactgatcttcagcatcttttactttcaccagcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctgacgtcccaatagatctggctttccacctagggaaggaatactcctgcctgcctgcctgcctggatcctgccagaggacggatgaagctggaaggaaccgccgtgctccgcgcccgctccccttccccgctcgttgcctggtcacaggccgcggctgggagcccgcccccccccactcgggagcgcgggcctcgtggtcagcgcctccgcggggagaaacaaaggaggcggcggcggctcaagggcagcgccagcagcgcacgcctccccctccggcggccgccacgtagcggggagcgcgctgcagctcgggcctggggcctcgcggcggcggcggcggctctaacgttatccgcgcccgccggcccgcggcgcggctgggggaggggacccgcgcgctttgtctggaggccccgcagctgtcatcgctctcccaccctcggcacgactgtgccgtcctcccccgtgcagcggcgcggcgcggcgcggcgcggcgcggcggcgcctcagccaggccccgccaagcctcctccccgcagcagccccgggtcccgggggcccacggcggggcgcggggggcccgcagcctccccgcagggaagccgggcgcacggagcatccccggccccgccgcgccgcgccgcgctgcgccacgcgcgacccagaaaagcaaggcaccgacttcaccgctcagatgctccgggcgcggatcctgcagagtctcccggctgcgcgcctcagctcagccacatccgaggggagggggcgcgggcgcgggcgcgggcgcgggcggctcggccgggggggaggggaggggaggcaccgcccacgcccaggccgcgcggggcccgccgggaacgcgtcctgcggggggcggcgtgcgcgccactcaccgtccgcggcgtggcgccttgggggtctcctgaggggaggcgggcgggaccgccgaggccgctctgcgtcaaagcccagcgcgccaaagccgggctccgcccacctcctcgccccggggcctgcggggctggggagcttctgagcaccgcctctcacggggcgcgggggtcccagggcctcgggtgccgccgcggcagccgcgctcggactgcaccccccctgggggacgggcacaaacaaacaaaggccggcgcgcctcgagaggcccgcagcgaggatcggcgaaggcgtcgtcgccgcagggagtgcggccggggtgacgagggcctcacgcctgagcggcgacgcaagccccgcccggcagcgttaatttcaaactgcgcgaccgtttctccggctccttgcgcggaggtgtggggggggagggccggacctcacaccggacgtagtaacacgttgggcgcgcgagccttccgcaattcaccaaacgcggttgcgcaagtgacgtaacgcggcctcgctatcccgggagggggagggcgcacgcacccggcgcaaaaggcgtgtcgaggcccttcccgccattggcggcggttaggacgtttgcgtaacggcctgacgtaggcggagacgcgttagtggggggaaggttctagaaaagcggcggtcccggctccagcggcagtagcagcggcgccgggtcctgtgtgcaggtctttcccccgcggcgcggtttctcgagcggcggctgctcttccccccccacagcgtcaggacgagtcccgtgcgtgtgcgtccgcggaggtctttctcatctcgctcggctgcgggaaatcgggctgaagcgactgagtccgcgatggaggtaacgggtttgaaatcaatgagttgttgttggcgagggcatggcgaggccgccgtggaagccggccaccgccgcggcgggcgggcgggcggagggagggagagcggccgcccgagcggcggttggcccgggggctctgagagggtcgttggcggaggcccgattgtccgaacgccttccccgccctcagccgcccggcgccatttccctgagccgggtgggggatggggcccggccgcggccagcggctcgtcctccgcaggggcgcgggagcccggccggcctggttcccccgcgggccgtgcgcggggccccggggcccggggcgtgtgcgcggcggagcgttcgttcgggcggccaccgggtccccctggagcggctctgcgcggctcccctccccctccctccctcctcccccccgggaaccggatttggcggccgccattttcccgtctccttgctcgcagcggtttttcgcgtcccgcactctgcggctctgcccgttcggccccttcccccccgcgtgggagccattagctggtttcctttgtccccggcccgttttggtggcgttccgagggggatctggggtgttttgagctctgatctcggagtcctgcgcacctcgttttaggcgctccgccctccttccccctcgacgaaatggcgccattgcgctcccgagccacacggtgtgcgcggccgggtgggggtggcggcggcctccgcaagggttcccggcgcaggccccgggcgaggagtgcgatccgggcctgagcctgctgctgaaggcagccttgcagcaaagaaaccattttacttccccgagctggctcctaattc